(19) United States
(12) Patent Application Publication
(10) Pub. No.: US 2022/0214367 A1
(43) Pub. Date: Jul. 7, 2022

(54) CALIBRATION METHODS AND COMPOSITIONS FOR BIOMOLECULE ANALYSIS

(71) Applicant: BOARD OF REGENTS, THE UNIVERSITY OF TEXAS SYSTEM, Austin, TX (US)

(72) Inventor: James Thomas BRENNA, Austin, TX (US)

(73) Assignee: BOARD OF REGENTS, THE UNIVERSITY OF TEXAS SYSTEM, Austin, TX (US)

(21) Appl. No.: 17/603,667

(22) PCT Filed: Apr. 14, 2020
(86) PCT No.: PCT/US2020/028075

§ 371 Date: Oct. 14, 2021

Related U.S. Application Data
(60) Provisional application No. 62/834,130, filed on Apr. 15, 2019.

(51) Int. Cl. G01N 33/92; G01N 33/96

(52) U.S. Cl. CPC G01N 33/92; G01N 2405/04; G01N 2405/02; G01N 33/96

(57) ABSTRACT

Calibration standards with known amounts of combinatorial biomolecules, such as GLs, are provided. In some aspects, calibration markers of the embodiments can be used to assess or quantitate the levels of a plurality of biomolecules in a test sample, such as by mass spectrometry.

This application claims the benefit of priority to U.S. Provisional Application No. 62/834,130, filed on Apr. 15, 2019, the entire contents of which are hereby incorporated by reference.

### BACKGROUND OF THE INVENTION

### 1. Field of the Invention

The present invention relates generally to the field of chemistry and biochemistry. More particularly, it concerns methods for analysis and quantitation of combinatorial biomolecules and calibration standards for the same.

### 2. Description of Related Art

Many biological moles are formulated from subunit constituents in a combinatorial fashion. As an example, acyl glycerolipids (GL) are made from a glycerol backbone with acyl groups esterified to one of three positions. As such, native GL represent a sort of combinatorial chemistry, where the open sites on the glycerol carry any of a usually large number of distinct acyl groups drawn from a fatty acid (FA) pool. GL molecular species reflect net acyl specificity of many different lipases and acyltransferases operating continuously to build and maintain particular functional GL profiles in distinct cellular and extracellular sites. Physicochemical and biological properties of lipid droplets and membranes depend on arrangement and structures FA in their component GL.

Acyl asymmetry has been assessed in GL using classic biochemical methods. For example, these methods employ enzymes to cleave acyl groups at a particular position with subsequent derivatization to methyl esters and analysis by gas chromatography (GC) or GC/MS to yield quantitative FA profiles at that position. Such methods were successful at establishing the non-random character of GL components. For instance, in many TAG samples, the sn-2 position is dominated by unsaturated FA, while the sn-1/3 positions are mostly saturated. An important and mysterious counterexample is human milk lipids, where the saturate palmitic acid (16:0, P) is predominantly found, unlike all other milks (e.g. cow's milk). Membrane lipids are highly specific, for instance with very long chain FA (e.g. 36:6n-3) in the sn-1 position and 22:6n-3 in the sn-2 position in retinal lipids. In most places, lipid molecular species are found within vast combinatorial distributions with general configurations favored in particular locales, e.g. saturated PL in lipid rafts.

Biology expends substantial free energy to maintain non-random GL profiles. While biochemical pathways known for synthesis of TG and PL, as well as their remodeling (replacement of one class of acyl groups with another), the principles governing, and mechanisms driving, acyl asymmetry are largely vague. This is at least in part because the full quantitative distribution of native GL species, the quantitative GL profile, is unknown in most systems.

The reference method (“gold standard” method) for calibration of molecular abundance is by addition of isotopologue of the target analyte. This is most commonly achieved by preparing by total synthesis each molecule of interest and adding each to the analyte mixture. It is practical for the pharmaceutical industry where it originated because the problem reduces to analysis of a finite set of metabolites, and total synthesis of the parent drug is available, and metabolites can be conveniently labeled. Alternatively, natural materials may be purified to prepare calibration curves and/or use the method of standard addition for calibration. All these methods require synthetic capabilities and/or purified standards. A method is needed to develop response factors, analogous to the routine in FA profiling (Brenna 2013).

A form of quantitative analysis is practiced for comparative experimental treatments. For instance, a control and experimental treatment of cells is implemented, and lipids extracted and analyzed identically. Ratios of corresponding feature signals between samples, defined by LC retention times and mass spectra, suggest whether the underlying concentrations from are responsive to the particular treatment. This strategy does not yield relative concentrations of analytes within a particular mixture. It is the quantitative GL profile, analogous to the quantitative FA profile, that is needed to understand detailed membrane composition, lipid droplet composition, and the way those distributions adjust to alterations in the supply of component fatty acids.

The basic problem is that no quantitative standards are available for the combinatorial-like mixtures found in all-natural GL. Quantitative analysis is generally accomplished using methods established for drug metabolites numbering 10 or so analytes (Shah et al., 2000), specifically use of calibration curves and isotopologues to determine response factors. These methods require macroscopically purified chemical standards or synthesis of isotopologues, expensive and often rare substances that require highly specialized chemical skills. Extension of this approach to thousands let alone millions of analytes is impractical.

While great strides have been made in identification of structure by mass spectrometry (MS), methods for quantitative analysis are limited to those established broadly in the pharmaceutical industry for small numbers of analytes, using calibration curves derived from purified analytes or isotopically labeled analytes. These methods are impractical for 1,000s to 1,000,000s of analytes encountered in combinatorial-like biological molecules, such as GLs and biological polymers. As such, methods for preparing standards of known relative and/or absolute concentrations of such molecules needed are needed.

### SUMMARY OF THE INVENTION

In a first embodiment there is provided calibration compositions of recombinant glycerlipids (GLs) comprising a mixture of GLs having a plurality of different FAs and/or polar head groups wherein there is a known proportion of each individual GL. In some aspects, the composition is essentially free of free glycerol and/or free fatty acids. In some aspects, the GLs comprise a triacylglycerol TAG mixture. In some aspects, the GLs comprise a phospholipid (PL) mixture. In further aspects, the PL mixture comprises PLs with phosphocholine, phosphoethanolamine and/or phosphoserine head groups. In some aspects, the GLs comprise a cardiolipin (CL) mixture. In some aspects, the compositions comprise at least 100 distinct GLs present in a known proportion. In some aspects, the compositions comprise at least 500 distinct GLs present in a known proportion. In some aspects, the compositions comprise at least 1,000 distinct GLs present in a known proportion. In some aspects, the compositions comprise 1,000 to 10,000 GLs present in a known proportion. In some aspects, at least one of the GLs is labeled. In some aspects, at least one of the FAs present in the GLs is labeled. In some aspects, the label is a isotopic label. In some aspects, the isotopic label is not radioactive.

In another embodiment, the present disclosure provides kits comprising two or more separately packaged compositions according to claim 1, where said separate compositions each comprise different known proportions the individual GLs.

In still another embodiment, the present disclosure provides spiked test samples comprising a first portion having an organic sample having an unknown level of GLs and a second portion having a mixture of GLs having a plurality of different FAs and/or polar head groups where there is a known proportion of each individual GL.

In yet another embodiment, the present disclosure provides methods of obtaining a quantitative GL profile of a test sample comprising performing mass spectrometry on a test sample and a calibration composition according to claim 1; and comparing the GL profile of the test sample to the calibration composition thereby obtaining the quantitative GL profile for the test sample.

In another embodiment, the present disclosure provides methods of obtaining a quantitative GL profile of a test sample comprising spiking a first portion of a test sample with a calibration composition according to anyone of claim 1; performing mass spectrometry on the first portion of the spiked test sample and a second portion of the test sample; and comparing the GL profile of the first portion of the spiked test sample to the second portion of the test sample thereby obtaining the quantitative GL profile for the test sample.

In still another embodiment, the present disclosure provides methods for measuring the amounts of a plurality combinatorial analytes in a test sample comprising: (a) measuring the quantitative profiles (QP) of substituent moieties of the combinatorial analytes; (b) reacting the substituent moieties to form a mixture combinatorial analytes in a manner that preserves the QP in a predictable way to generate a calibration standard; (c) analyzing the calibration standard and a test sample by the same chromatography and/or spectrometry analysis method; and (d) comparing the analysis of the calibration standard to the analysis of the test sample to determine the amounts of a plurality combinatorial analytes in the test sample. In some aspects, the calibration standard and the test sample are analyzed separately. In some aspects, the calibration standard and the test sample are mixed prior to analysis. In some aspects, the methods further comprise adding an internal standard that is a known quantity of a substituent moiety before the reacting step (b). In some aspects, the internal standard is labeled. In further aspects, the label is an isotopic label. In some aspects, the internal standard was not previously present in the sample of substituent moieties of the combinatorial analytes of (a).

In yet another embodiment, the present disclosure provides methods of producing a calibration standard comprising: (a′) obtaining a mixture of plurality combinatorial analytes and reacting the mixture to produce the substituent moieties of the combinatorial analytes; (a) measuring the quantitative profiles (QP) of the substituent moieties of the combinatorial analytes; and (b) reacting the substituent moieties to form a mixture combinatorial analytes in a manner that preserves the QP in a predictable way to generate a calibration standard. In some aspects, the methods further comprise adding an internal standard that is a known quantity of a substituent moiety before the reacting step (b). In further aspects, the internal standard is labeled. In still further aspects, the label is an isotopic label. In some aspects, the internal standard was not previously present in the sample of substituent moieties of the combinatorial analytes of (a). In some aspects, the methods further comprise analyzing the calibration standard by a chromatography and/or spectrometry analysis method. In some aspects, the methods further comprise spiking the calibration standard with a known quantity of at least first combinatorial analyte. In some aspects, the plurality of combinatorial analytes comprises at least one phospholipid group. In further aspects, the at least one phospholipid group is glycerophosphatidylcholine (GPC). In some aspects, the plurality of combinatorial analytes comprises at least one fatty acid. In further aspects, the plurality of combinatorial analytes comprises 2 or 3 fatty acids. In some aspects, reacting comprises contacting the mixture with at least one reagent suitable to effect acylation. In further aspects, reacting comprises contacting the mixture with 1-ethyl-3-(3-dimethylaminopropyl)carbodiimide) (EDC) and 4-dimethylaminopyridine (DMAP). In some aspects, reacting further comprises contacting the mixture with a solid support, such as diatomaceous earth.

In another embodiment, the present disclosure provides methods for measuring the amounts of a plurality combinatorial analytes in a test sample comprising: (c′) obtaining a calibration standard comprising a plurality of combinatorial analytes, wherein the proportion of each of the plurality of combinatorial analytes in the standard it known; (c) analyzing the calibration standard and a test sample by the same chromatography and/or spectrometry analysis method; and (d) comparing the analysis of the calibration standard to the analysis of the test sample to determine the amounts of a plurality combinatorial analytes in the test sample. In some aspects, the calibration standard was produced by a method of the present disclosure. In some aspects, the analyzing is by chromatography, such as gas chromatography (GC) or GC-Flame-ionization detection (GC-FID). In some aspects, the analyzing is by spectrometry. In some aspects, the analyzing is by Electrospray Ionization Mass Spectrometry (EI-MS). In some aspects, the analyzing is by GC/MS or Gas chromatography-electron ionization mass spectrometry (GC-EIMS). In some aspects, the plurality of combinatorial analytes comprises a GL mixture. In some aspects, the calibration standard comprises at least 100 distinct GLs present in a known proportion. In some aspects, the calibration standard comprises at least 500 distinct GLs present in a known proportion. In some aspects, the calibration standard comprises at least 1,000 distinct GLs present in a known proportion. In some aspects, the calibration standard comprises 1,000 to 10,000 GLs present in a known proportion. In some aspects, the plurality of combinatorial analytes comprises a TAG mixture. In some aspects, the plurality of combinatorial analytes comprises a CL mixture. In some aspects, the substituent moieties of the combinatorial analytes are fatty acids and a non-combinatorial reactant is glycerol. In some aspects, the plurality of combinatorial analytes comprises a polypeptide mixture. In some aspects, the plurality of combinatorial analytes comprises a carbohydrate mixture. In some aspects, the plurality of combinatorial analytes comprises a combination of carbohydrate and lipid mixtures. In some aspects, the plurality of combinatorial analytes comprises carbohydrate and protein mixture. In some aspects, the plurality of combinatorial analytes comprises a lipid and protein mixture.

As used herein, “sample” or “liquid samples” can refer to extracts from tissues or other biological specimens (e.g., extracts comprising proteins and metabolites) obtained by contacting tissue or biological specimen with a solvent according to the embodiments. In some aspects, a sample can be an extract from a non-biological specimen, such as the surface on an object (e.g., a forensic sample).

As used herein, “essentially free,” in terms of a specified component, is used herein to mean that none of the specified components has been purposefully formulated into a composition and/or is present only as a contaminant or in trace amounts. The total amount of the specified component resulting from any unintended contamination of a composition is therefore well below 0.01%. Most preferred is a composition in which no amount of the specified component can be detected with standard analytical methods.

As used herein in the specification and claims, “a” or “an” may mean one or more. As used herein in the specification and claims, when used in conjunction with the word “comprising”, the words “a” or “an” may mean one or more than one. As used herein, in the specification and claim, “another” or “a further” may mean at least a second or more.

As used herein in the specification and claims, the terms “conduit” and “tube” are used interchangeably and refer to a structure that can be used to direct flow of a gas or liquid.

As used herein in the specification and claims, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows three acyl glycerolipid (GL) classes: triacylglycerol (TAG), phospholipid (PL), drawn here as a phosphatidyl choline, and cardiolipin (CL). The glycerol backbone is circled. TAG, PL, and CL have 3, 2, and 4 acyl groups esterified, respectively, shown as specific hydrocarbon chains.

FIG. 2 shows acyl-CoA metabolic reactions (see Cooper et al. 2015). Acyl specificity is determined by the net effect of a complex organismal-wide metabolic network as well as highly specialized reactions in cells. It is usually mediated at esterification steps in the pathways to GL synthesis, as well as in direct transfer reactions such as lecithin-cholesterol acyl transferase (LCAT, not shown).

FIG. 3 shows intra- and interesterification of a specific TAG (SOL) results in every combination of TAG in a final product mixture randomly distributed among all possible isomers. S=stearic acid (18:0); O=oleic acid (18:1n-9); L=linoleic acid (18:2n-6). The hydrolyzed FA profile from the parent is the pool of FA. The randomized sn-1, sn-2, sn-3, and total FA profile are all identical to this FA profile and the various FA and GL (TAG here) are in fixed ratio to one another (Sreenivasan, 1978).

FIGS. 4A & 4B show gas chromatograms of FAME from triglycerides of (FIG. 4A) native fCO and (FIG. 4B) fCO+methyl oleate (18:1) after randomization. 18:1 is present at the expected ratio of 3:1 in the randomized TAG.

FIGS. 5A & 5B shows. ESI-MS1 (infusion) spectra of the TAG region of A) native fCO and B) randomized fCO and methyl 18:1 in the ratio of 3:1, demonstrating that the randomization chemistry is operable. Major triglycerides in fCO show various combinations of the major fatty acids (FA) in proportions reflecting acyl specificity. The randomized TAG in B) show different proportions of the FA C8-12 from fCO and new TAG with 18:1. 8=8:0; 10=10:0; 12=12:0; 18:1 is oleic acid derived from methyl oleate. TAG a-b-c reflect component FA a, b, and c which may be any combination of the four major FA (8:0, 10:0, 12:0, 18:1) and all regioisomers (sn-1,2,3).

FIGS. 6A & 6B show (FIG. 6A) mixture of equal parts fCO and soy oil. Soy oil composition is 55% 18:2. fCO is as in FIG. 5. (FIG. 6B) Interesterified fCO and soy oil. The boxed central region shows the various TAG with 18:2 and fCO FA.

FIG. 7 shows zoomed-in spectrum of TAGs w/ 16:0 and 18:1.

FIG. 8 shows full spectrum of TAGs w/ 16:0 and 18:1.

FIG. 9 shows full spectrum of PC w/ 16:0, 18:1, and 18:2.

FIG. 10 shows combinatorial PC zoomed-in spectrum of PC w/ 16:0, 18:1, and 18:2. Three fatty acids (16:0, 18:1, 18:2) combined in equimolar proportion used to synthesize a combinatorial PC, analyzed by infusion ESI-MS1. Six protiated and six sodiated PCs are found all in the proper combinatorial proportions. 16:0-16:0; 16:0-18:2; 16:0-18:1; 18:2-18:2; 18:2-18:1; 18:1-18:1 (protiated) all are in the correct proportions (1:2:2:1:2:1 in order of increasing mass), considering 18:1-18:2 is isobaric with Na+16:0-18:1.

FIG. 11 shows expanded zoomed-in spectrum of PC w/ 16:0, 18:1, and 18:2.

### DESCRIPTION OF ILLUSTRATIVE EMBODIMENTS

### I. The Present Embodiments

The present disclosure, in some aspects, provides for calibration standards that can be used for the analysis of combinatorial biomolecules, such as lipids and biological polymers. Such methods can provide calibration standards (or panels of calibration standards) having known amount do an array of biological molecules of interest, such as GLs. Such strands can be used to efficient assess the content of the indicated biological molecules in test sample, such as by mass spectrometry. Moreover, the standards and methods provided herein can be used to quantitate the proportions or absolute amounts of a plurality of biological molecules of interest in the test sample. Thus, in some embodiments, the present disclosure provides methods for quantitative analysis of acyl GL continuants of sample by comparison to a calibration standard. In some aspects, the method comprises the conversion of signals indicative of specific GL to quantitative information specific to the original sample.

### II. Acyl Glycerolipid (GL) and Analysis Thereof

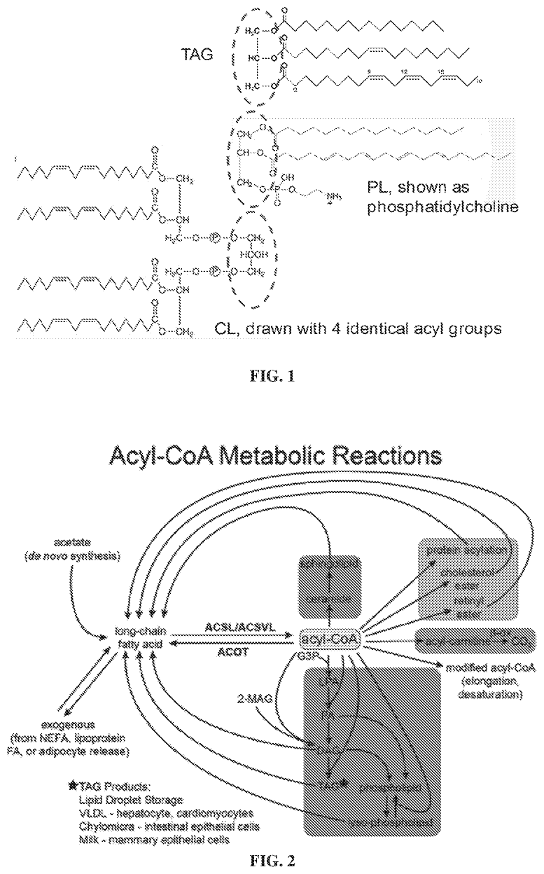

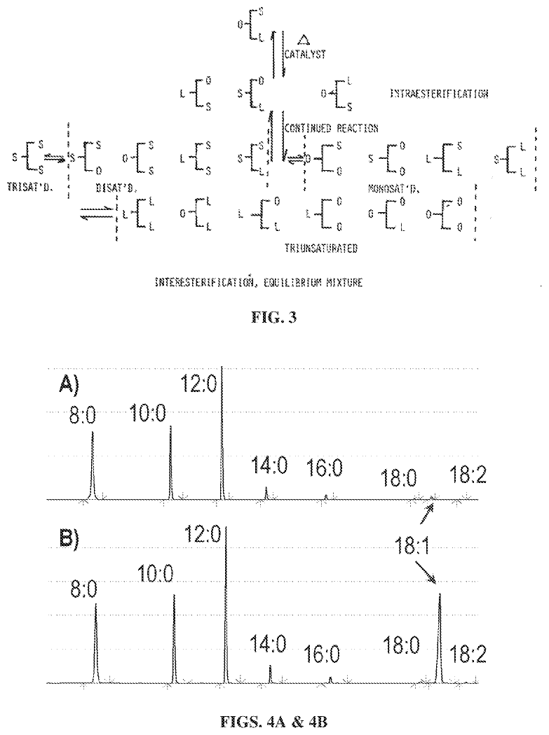

In some aspects, the GL are triacylglycerols (TAG), phospholipids (PL), and/or cardiolipin (CL), as show FIG. 1. PL are further divided into numerous classes defined by various polar headgroups such as phosphatidylcholine (PC), phosphatidylserine (PS) and phosphatidylinositol (PI). Each GL can be considered as synthesized from a pool of fatty acids (FA) and a glycerol backbone. In some aspects, the present disclosure provides methods of characterizing mixtures of acyl phospholipids. In some aspects, the present disclosure provides method of characterizing mixtures of ether phospholipids.

Natural lipids are inherently complex chemical mixtures and may include proteins and amphipathic compounds that modify physicochemical properties. Even at the gross nutritional level, interesterified fats have become a topic of current controversy over possible adverse metabolic effects (Mensink et al., 2016). Without being bound by any theory, in cells and tissue, GL profiles are maintained by a complex series of reactions mediated by at least 100 proteins involved in transport, biosynthesis, and maintenance (FIG. 2; Cooper et al. 2015).

Quantitative FA profiles are a key to lipid composition. Traditional techniques, particularly hydrolysis by lipases and phospholipases with subsequent lipid class separation by TLC, conversion of acyl and lysoGL to FAME and analysis by quantitative GC-FID or GC/MS have established that biology expends substantial free energy in maintaining non-random distributions of acyl groups on GL.

The issue also recognized most clearly in the lipidomics era is that the GL profiles, that is which GL acyl pairs, triplets, or quartets are favored at the expense of others, cannot be determined by previous methods. In parallel, advances in chemical computing have established the importance of lipid structure in membranes and lipid droplets but suffer from a lack of quantitative empirical information about GL structure. Inputs to membrane simulations are estimates. As such methods for producing accurate empirical data on membrane lipid composition may dramatically enhance the accuracy of modeling and understanding of membrane function.

Specific low concentration fatty acids (FA) are most potently bioactive and relevant for human health (Leng et al., 2018). For instance, omega-3 DHA (docosahexaenoic acid) widely acknowledged to be of importance to infant neural development is present in breastmilk at a mean of about 0.32% of FA, while the omega-6 arachidonic acid is 0.47% of FA (Brenna et al., 2007). While they are more efficiently delivered to the brain when components of PL rather than TAG (Wijendran et al., 2002; Liu et al., 2014), little is known of the molecular species, transport details, and how they access the brain. Other examples are the conjugated linoleic acids (CLA) and branched chain FA in cow's milk. With respect to molecular biology, the FA profiles of adipose from white, brown, and beige fat are well known but the quantitative molecular profiles are poorly characterized, especially for minor FA. Targeted methods can establish the coupling of minor FA with other acyl groups in GL, but without quantitative methods the relative importance of the molecular species is vague and depends on secondary standards, at best. In some aspects, the present disclosure provides methods for preparing standards of known relative concentrations of combinatorial analyte mixtures. In some aspects, mixtures of arbitrary numbers of GL all of known relative concentration are synthesized. In some aspects, these mixtures of known quantitative GL profile incorporate known amounts of internals standards. In some aspects, the GL are isotopically labeled. In some aspects, the mixtures are made on an experiment-wise basis, that is, specific to a particular lipid preparation. The approach is based on acyl randomization by interesterification. At equilibrium, the largely random distribution of acyl groups produces a mixture with known GL profile that can be calculated from the FA profile.

In some embodiments, the present disclosure provides calibration compositions of recombinant glycerolipids (GLs) comprising a mixture of GLs having a plurality of different fatty acids (FAs) and/or polar head groups wherein there is a known proportion of each individual GL. Fatty acids contemplated for use in the methods of the present disclosure include saturated fatty acids, unsaturated fatty acids, short-chain fatty acids, medium-chain fatty acids, long-chain fatty acids, and/or very long chain fatty acids. Non-limiting examples of fatty acids include linoleic acid, stearic acid, oleic acid, arachidonic acid, docosapentaenoic acid, docosahexaenoic acid, α-linolenic acid, rumenic acid, palmitic acid, mead acid, eicosapentaenoic acid, eicosatrienoic acid, erucic acid, adrenic acid, octacosaoctaenoic acid, tetracosahexaenoic acid, and isomers and isotopologues thereof.

Atoms making up the fatty acids described herein may be replaced with any isotopic form of such atoms. Isotopes, as used herein, include those atoms having the same atomic number but different mass numbers. By way of general example and without limitation, isotopes of hydrogen include tritium and deuterium, isotopes of carbon include ¹³C and ¹⁴C, and isotopes of oxygen include ¹⁶O, ¹⁷O, and ¹⁸O.

In some aspects, the GLs comprise a phospholipid (PL) mixture. The phospholipids comprise a head group. Non-limiting examples of PL head groups include hydrogen, phosphocholine, phosphoserine, phosphoglycerol, phosphoethanolamine, inositol, myo-inositol 4,5-biphosphate, phosphatidylglycerol, phosphatidylglycerolphosphate, and/or 1,3-bis(sn-3′-phosphatidyl)-sn-glycerol (cardiolipin).

### III. Mass Spectrometry (MS), Chromatography, and Nuclear Magnetic Resonance Spectroscopy (NMR)

Mass spectrometry (MS) is an analytical technique that ionizes chemical species and sorts the ions based on their mass-to-charge ratio, essentially measuring the masses within a sample. Mass spectrometry is used in many different fields and is applied to pure samples as well as complex mixtures.

A mass spectrum is a plot of the ion signal as a function of the mass-to-charge ratio. These spectra are used to determine the elemental or isotopic signature of a sample, the masses of particles and of molecules, and to elucidate the chemical structures of molecules and other chemical compounds.

In a typical MS procedure, a sample, which may be solid, liquid, or gas, is ionized, for example by bombarding it with electrons. This may cause some of the sample's molecules to break into charged fragments. These ions are then separated according to their mass-to-charge ratio, typically by accelerating them and subjecting them to an electric or magnetic field: ions of the same mass-to-charge ratio will undergo the same amount of deflection. The ions are detected by a mechanism capable of detecting charged particles, such as an electron multiplier. Results are displayed as spectra of the relative abundance of detected ions as a function of the mass-to-charge ratio. The atoms or molecules in the sample can be identified by correlating known masses (e.g. an entire molecule) to the identified masses or through a characteristic fragmentation pattern.

One of skill in the art recognizes that there are many methods for ionization and mass selection that may be employed in mass spectrometry. Non-limiting examples of ionization techniques include electron ionization (EI), fast atom bombardment (FAB), chemical ionization (CI), atmospheric-pressure chemical ionization (APCI), electrospray ionization (ESI), matrix-assisted laser desorption/ionization (MALDI), inductively coupled plasma (ICP) ionization, photoionization, glow discharge, field desorption, thermospray, desorption/ionization on silicon (DIOS), Direct Analysis in Real Time (DART), secondary ion mass spectrometry (SIMS), spark ionization, and thermal ionization (TIMS). Non-limiting examples of analyzers for mass selection include sector field mass analyzers, time-of-flight (TOF) analyzers, and quadrupole mass analyzers.

Chromatographic separation methods may be coupled with mass spectrometry methods to enhance resolution and mass determination. Non-limiting examples of separation techniques that may be coupled to MS include gas chromatography, liquid chromatography, capillary electrophoresis, and ion-mobility spectrometry.

Nuclear magnetic resonance (NMR) spectroscopy relies on the NMR phenomenon in which atomic nuclei placed in a static magnetic field interact with an electromagnetic wave having a frequency specific to the atomic nuclei. An apparatus that utilizes said phenomenon for measuring a test sample at an atomic level is referred to as an NMR spectrometer. NMR spectrometers have been used for analyzing materials, including organic compounds (such as, for example, chemical agents and agricultural chemicals), polymeric materials (such as, for example, vinyl and polyethylene), and biological materials (such as, for example, nucleic acids, proteins, and fatty acids). NMR spectroscopy, for example, the molecular structure of a sample can be identified.

In general, the NMR measuring apparatus includes a control computer, an RF signal transmitter, an NMR signal detector (probe), a static magnetic field generator (a superconductive magnet), an NMR signal receiver, and other components. In some cases, however, the NMR measuring apparatus may refer to a part of the NMR measuring apparatus including some of the above-listed components. For example, a part corresponding to a spectrometer including the control computer, the RF signal transmitter, and the NMR signal receiver may be referred to as the NMR measuring apparatus. In a typical NMR measurement, a radio frequency signal (RF transmission signal) used for the NMR measurement is generated in the transmitter and supplied to a transmitting and receiving coil in a probe. This generates an electromagnetic wave that causes a resonance absorption phenomenon in nuclei to be observed within the sample. Then, an NMR signal (RF reception signal) induced in the transmitting and receiving, coil is sent to the receiver, and a spectrum of the received signal is analyzed.

The following abbreviations are used throughout the present disclosure: CACI, covalent adduct chemical ionization, which is a method to determine double bond position in fatty acid methyl esters (FAME); GL, glycerolipid; TAG, triacylglycerol; PL, phospholipid; CL, cardiolipin; n:db n-x, n=number of C; db=number of double bonds; x=location of the last db. E.g., 22:6n-3 is docosahexaenoic acid (DHA), 22 C, 6 db, last db located 3 C atoms from the methyl terminus; FA, fatty acid; FFA, free fatty acid; PUFA, polyunsaturated fatty acid; GL profile, glycerolipidome; nGL, native GL; rGL, randomized GL; FID, flame ionization detector; GC, gas chromatography; MS, mass spectrometry.

### III. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Prophetic Example 1—Quantitative Glycerolipidomics

Analytical methods for rigorously accurate quantitative analysis of acyl glycerolipid (GL) profiles will be developed that will enable a) quantitative evaluation of net acyl specificity in GL pools under various treatments and conditions, b) accurate modeling of lipid droplets and membrane behavior to understand how and why acyl specificity is crucial, c) biological tolerance for deranged acyl specificity and the physiological consequences. Progress has been made in GL structural analysis with modern liquid chromatography-electrospray ionization mass spectrometry (LC/MS). Quantitative GL molecular species analysis currently relies on isotopologue-based internal standards and/or calibration curves, borrowed from pharmaceutical applications. They require specialized chemical expertise, time, and expense, and typically calibrate 10-20 metabolites using purchased standards. Methods that employ comprehensive standards specific to sample types are required. A strategy to create a mixture of an arbitrary number of GL molecules, hundreds to millions of analytes, all of known concentration will be developed.

Native GL as prepared from biological tissue, cells, or fluids resemble a mixture made by imperfectly randomized combinatorial chemistry. Acyl (R) groups occupy 2, 3, or 4 positions in the cases of phospholipids (PL), triacylglycerols (TAG), or cardiolipins (CL), respectively. The fatty acid (FA) profile that defines the probabilities of each member of the combinatorial pool will be quantitatively characterized by hydrolyzing and quantifying the FA profile by standard means. The concentration of any component of the combinatorial library of PL, TAG, or CL will be defined in a random combinatorial mixture by the product of the abundances of the FA in each particular GL. Rigorous methods to synthesize libraries of combinatorial GL will be developed, all with quantitatively defined profiles referred to as “universal-randomized GL”, u-rGL. These libraries will then be used as standards to calibrate native GL profiles using response factors to calibrate similar native GL mixtures analyzed by any desired method. Methods to measure the degree to which native GL mixtures deviate from a random distribution will be developed. Biology expends substantial free energy to create and maintain by various processes GL profiles with acyl distributions far from random. Desired GL mixtures will be isolated, and protocols developed to randomize acyl groups, then analyze native GL (nGL) and random GL (rGL) side-by-side, calibrated against u-nGL. Quantitative biological indexes of non-random character will be developed consisting of ratios and proportions of specific GL species. Yeast and retinal lipid studies, representing simple and complex eukaryotic GL systems, respectively, will generate data on how GL profiles and indexes adjust to exogenous FA treatment, growth conditions, and genetic manipulation. The insights gained will may lead to discovery of diseases of GL profile dysfunction, and support therapies for their amelioration.

Combinatorial GL. GL are molecules that can be generated by combinatorial chemistry of a fatty acid pool and the three positions in glycerol. The distribution of distinct GL molecules generated by combinatorial chemistry is random, modified only by minor equilibrium chemical effects. Random means all outcomes are equally probable. While chemical effects such as steric hindrance could, in principle, influence the GL profile from strictly random, no such effects have been reported or are expected based on the 3D structure of glycerol. If equilibrium effects were to be found, they will be subtle and may be handled by empirical coefficients developed from isotopologue sentinel experiments (below).

Fatty acid (FA) profile by high resolution GC-FID. The FA profile of a hydrolyzed GL can be measured with high accuracy and precision by high resolution capillary GC coupled to FID.

Randomized GL (rGL). While methods for randomization of acyl groups on TAG are known (Gibon, 2011) and analytical scale randomization using chemical and biochemical catalyst has generally been done on a research basis for modification of fat properties, the precise degree of randomization has not been a priority for industrial processes. Tailoring conditions for analytical scale standards and applications remains to be defined.

Signal ratios for relative quantification. Discovery and quantitative metabolomics may apply two or more treatments to a system, for instance a cell culture, then ratios of signals are calculated to establish changes related to treatment, anchored to measurements of internal standards.

Quantitative profiling. Quantitative profiling is the quantitative analysis of relative proportions of a class of analytes. Familiar examples are FA profiles and amino acid profiles.

Combinatorial chemistry was developed as a rapid method for synthesis of molecular libraries, initially for pharmacology (Liu et al., 2017) and now for many applications (De Lue, 2001). A form of combinatorial chemistry will be used to create calibration mixtures, recognizing that probabilities define composition when equilibrium is reached.

Reference to “concentrations” in GL refer to a molar proportion of the whole of GL, analogous to the mole or weight percent used in FA profiles. Profile concentrations can be put on an absolute concentration basis (e.g. mmol POP per ml plasma, where POP is a TAG with palmitic-oleic-palmitic acyl groups) using an internal standard of known molar concentration. Importantly, as shown below, concentration ratios and proportions of subsets of GL can be constructed to avoid any need to measure a total GL profile when not needed.

The abundance of each molecular species in a randomized GL is a product of their abundances in the FA profile. This principle enables calculation of the GL profile of an arbitrary number of molecular species.

For instance, consider U.S. retail cow's milk FA profile (O'Donnell-Megaro et al., 2011), assuming 100% as TAG, shown in Table 1. The left 3 columns are the empirically measured FA profile of retail cow's milkfat with the 22 most abundant FA. A randomized TAG (rTAG) would have 22³=10,648 distinct molecular species. In the right side, the abundance of TAG with the 20 combinations of FA is calculated regardless of regiospecificity (sn-1,2,3). (Each of the 6 possible isomers (POS, PSO, OPS, SPO, OSP, SOP) are equally probable in an rGL and the concentration of each would be the %/6=0.727/6=0.121.) Column TAG lists triplets representing TAG with FA corresponding to the line and the next 2 lines, for instance the 16:0 row's TAG is POS with predicted abundance=27.99% x23.90% x10.88%=0.727%; the next is OSM with abundance=23.90% x10.88% x9.83%=0.250%, etc, given in 5^{th} column. In the last column is the abundance ratio of consecutive rows, for instance the abundance ratio of POS to OSM=0.727×0.250=2.91, the abundance ratio of OSM to SMB is 5.73, etc. The ratio of abundances emphasizes two key points: (1) for the purpose of validation of the present randomization procedure, ratios of abundances can be used at all abundance levels; (2) low abundance GL, TAG in this case, which will include minor and highly bioactive FA, can be measured. For instance, 9cis-11trans-18:2, known as rumenic acid (R), has potent anticarcinogenic activity in rodent models (Kramer et al., 1998; Jensen and Lammi-Keefe, 2001). In U.S. retail milk the abundance is 0.57% of FA. With a standard mixture of defined GL profile, a profile of rumenic acid can be determined quantitatively from a lipid extract and suitable MS/MS method.

TABLE 1
FA Profile of US retail cow's (bovine) milk (O'Donnell-Megaro
et al., 2011) with calculated partial TAG profile for triplets
of consecutive FA. TAG predicted abundances are for all regioisomers,
e.g. POS abundance is a sum of abundances for the six possible
regioisomers for sn-1,2,3 as POS, PSO, OPS, SPO, OSP, SOP.
The final column is a ratio of TAG abundances for consecutive
rows, e.g. POS to OSM, OSM to SMB, etc.

| FA | Abbr | % | TAG | % | Ratio |
|---|---|---|---|---|---|
| 16:0 | P | 27.99 | POS | 0.727829568 | 2.91 |
| 9c18:1 | O | 23.90 | OSM | 0.250410816 | 5.73 |
| 18:0 | S | 10.88 | SMB | 0.043690925 | 3.11 |
| 14:0 | M | 9.63 | MBL | 0.014054985 | 3.33 |
| 4:0 | B | 4.17 | BLLa | 0.004217955 | 1.65 |
| 18:2n-9 | L | 3.50 | LLaCapric | 0.002559095 | 1.65 |
| 12:0 | La | 2.89 | LaCapricCapro | 0.001550080 | 1.86 |
| 10:0 | Capric | 2.53 | CapricCaproPo | 0.000831358 | 1.74 |
| 6:0 | Capro | 2.12 | CaproPoV | 0.000476470 | 1.84 |
| 16:1n-7 | Po | 1.55 | PoVCapril | 0.000258463 | 1.72 |
| 11t18:1 | V | 1.45 | VCaprilPen | 0.000150075 | 1.63 |
| 8:0 | Capril | 1.15 | CaprilPenMo | 0.000092115 | 2.02 |
| 15:0 | Pen | 0.90 | PenMoR | 0.000045657 | 1.73 |
| 14:1n-9 | Mo | 0.89 | MoR10tO | 0.000026380 | 1.78 |
| 9c11t18:2 | R | 0.57 | R10tOMar | 0.000014820 | 1.14 |
| 10t18:1 | 10tO | 0.52 | 10tOMar12tO | 0.000013000 | 1.27 |
| 17:0 | Mar | 0.50 | Mar12tOLn | 0.000010250 | 1.72 |
| 12t18:1 | 12tO | 0.50 | 12tOLnE | 0.000005945 | 1.79 |
| 18:3n-3 | Ln | 0.41 | LnEAd | 0.000003329 | 2.93 |
| 9t18:1 | E | 0.28 | EAdA | 0.000000353 | 4.67 |
| 20:4n-6 | Ad | 0.14 | | | |
| 20:0 | A | 0.09 | | | |

u-rGL. Universal-randomized GL (u-rGL) standards will be developed to serve as robust working and international standards enabling interlaboratory calibrations, including internal reference isotopologue sentinels as described herein. u-rGL will provide RF for calibration of native GL abundances. Calibrated GL profiles of arbitrary depth into the acyl glycerolipidome will be possible, or from any range of specific GL or specific FA.

Using experiment-wise measurements, factors will be developed that describe the degree to which biology maintains GL profile asymmetries in health and disease.

n-GL. As noted, biology maintains multiple systems for maintenance of GL profiles far from random. Validated protocols will be developed to randomized GL samples on an experiment-by-experiment (experiment-wise) basis so to enable analysis of the nGL aside the rGL. The ratio of any particular GL in a nGL to the corresponding rGL will be a quantitative measurement of biological network's bias toward or away from equilibrium. It is envisioned that this ratio will be an element of a chemistry-based index to describe the biological principle underlying the synthesis and maintenance of GLs, be they TAG, PL, or CL. The component measurements will have the distinct theoretical advantage of being related to a chemical reference point, namely equilibrium. In this regard, chemical thermodynamic principles may become an important tool for evaluating membranes and TAG droplets, needed to explain the empirically based deviations from lowest entropic states. Discovery of diseases of acyl specificity dysfunction caused by defects in GL synthesis or remodeling or maintenance levels are envisioned.

Isotopologue sentinels are a key innovation to provide positive proof of randomization. The concept is described in throughout the present disclosure.

It is envisioned that the developments described herein will fill current gaps in the chemistry and biophysics of membranes. Proper modeling of membranes requires quantitative profiles of PL in real membranes. The current state of technology enables such measurements, at most, only for major PL without resort to stereospecific synthesis.

Diseases of deranged fatty acid profiles are well known, for example Zellweger's syndrome (Gronn et al., 1990; Martinez et al., 1990; Martinex et al., 1992) and adrenoleukodystrophy (ALD) (Martinez et al., 1992; Christensen et al., 1989), and therapeutic tests are based on the measurement of quantitative fatty acid profiles. Quantitative fatty acid profiles to detect defects in fatty acid synthesis. Presently there is a lack of methods to detect defects in PL profiles.

Diseases of acyl asymmetry deficit are likely to be mechanistically found in PL and CL constituting membranes and in part revealed by TAG. The discoveries of defects in the net acyl asymmetry will lead to protein and gene defects, and therapies to correct them, for instance by drugs targeted at the relevant proteins. As with the FA conditions, the GL profiles will be measured to evaluate how they can be returned to normal. It is further envisioned that acyl asymmetry derangement is a primary cause of membrane dysfunction and likely is a common secondary factor. Functional deficits due to molecular defects in membrane protein structure are likely to be accompanied by compensatory changes in GL profiles, as well as other changes in membrane constituents. Aggregates of self-assembled microdomains of lipids and proteins known as lipid rafts have higher saturated fatty acid containing PL than the bulk membranes and appear to depend on coalescence of a specific GL profile.

Fatty acid methyl ester (FAME) analysis remains the most comprehensively evaluated method for double bond (db) positional determinations in FAME (Van Pelt et al., 1999; Lawrence and Brenna, 2006; Michaud et al., 2002; Van Pelt et al., 1999). This method has been adapted this method for infusion (shotgun) MS/MS (Xu and Brenna, 2007). Methods for qualitative analysis are available as the present standard development for GL will apply to all methods and including all non-MS methods, e.g. NMR.

The calibration strategy will be robust to many types of overlaps. For instance, isomers of linoleic acid are common in the diet with db at positions and geometries (9z, 12z), (9z, 11e), (10e, 12z

As quantitative profiling applies directly to all lipid containing samples, the present methods may be employed with human/mammalian blood and tissue as well as plant and microbial samples. Plant and microbial kingdoms generate a vast array of lipids seldom seen in humans.

In practice, analysis will proceed as follows. Isolate a GL fraction—TAG, PC, PE, CL, etc—to be quantitatively analyzed. Create a FAME mixture, from an aliquot of the nGL, for quantitative analysis of FA profile to quantitatively characterize the FA pool, and then calculate the profile of rGL. Create a randomized GL (rGL) from an aliquot of the nGL. Analyze rGL, nGL, and a suitable u-rGL by any desired method. The u-rGL will be used as a calibration reference for nGL and rGL, and serve as a base for inter-laboratory comparisons. Asymmetry factors describing the specific deviations from randomness will be calculated from ratios of specific, or groups of GL, in the nGL compared to the rGL.

Protocols will be developed and validated to support this strategy, and real-world problems will be investigated that will enable further refinement of the method while discovering applications and developing non-randomization factors to describe net deviations from random character of biological importance.

Methods will be developed to reliably randomize GL classes PL, TAG, and CL on lab scale; develop international standards for major classes of GL identified on the basis of their known FA profiles.

Interesterification/randomization is an industrial process used to alter the properties of oils, for TAG (Gibon, 2011) and for PL (Chojnacka, 2012). The present methods employ GL randomization for creation of analytical standards. Existing industrial methods for TAG interesterification have been used for achieving random TAG. Existing methods for PL interesterification are focused on incorporating high value FA, e.g. omega-3 EPA and DHA, into PL that otherwise would not contain them, e.g. soy lecithin. Existing lab scale methods for PL synthesis focus on regiospecific synthesis. Neither goal is specific to randomization. TAG and PL will be approached separately.

### Example 2—Randomization by NaOCH3 Used at Catalytic Levels

NaOCH₃ is one of a dozen chemical reagents successfully employed for TAG randomization/interesterification (Sreenivasan 1978). One or more FA not native to a fat, such as isotopologue standards, was be added quantitatively to the final randomized GL. Briefly, three starting substances were used to demonstrate the chemistry: fractionated coconut oil (fCO) and supermarket soy oil, and methyl octadecenoate, 18:1. Four grams total of oils were blended, dried with NaSO₄, flushed with N₂ and treated with NaOCH₃ at 90° C. for 80 minutes.

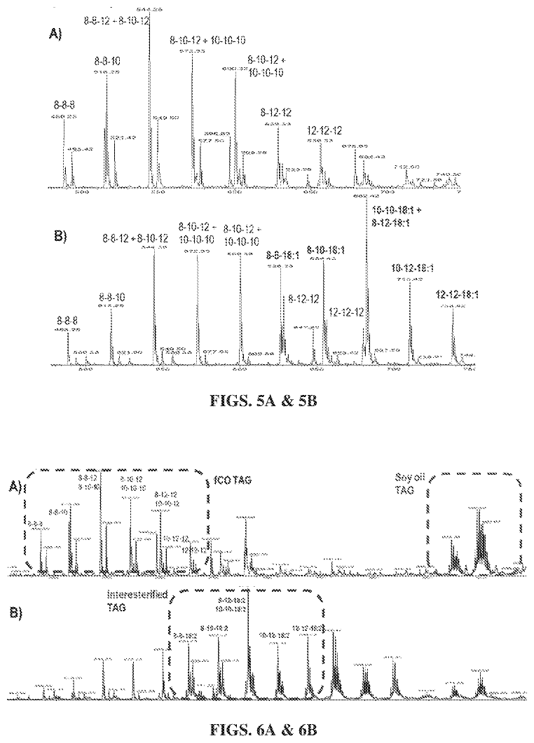

FIG. 4 is a GC-FID analysis of fatty acid methyl esters (FAME) prepared by conversion of TAG to FAME (A) fCO and (B) fCO+methyl 18:1 was blended at about 3:1 by weight. fCO is composed of TAG with three main FA, 8:0, 10:0, and 12:0. A2(B) reveals that the calibrated area percent of [18:1] is about 25% of the [8:0+10:0+12:0] in the randomized TAG, i.e. the expected 3:1 ratio. FIG. 5 is an infusion ESI-MS1 spectrum of the TAG region of the same samples. (A) shows the that the major TAG present in the fCO are the various combinations of the three main FA. (B) shows that the 18:1 interesterified into the TAG and is found in combination with each possible combination of the 3 fCO FA. Importantly, these data directly demonstrate that any FA, including isotopologue standards, can be interesterified into a TAG.

FIG. 6 shows interesterification of fCO and commodity soy oil. Focus is directed to the main component of soy, linoleic acid (18:2). (A) An equal mixture of the two oils was analyzed by infusion ESI-MS. TAG from fCO and at higher mass the soy TAG comprised of FA mainly comprised of C16-18. (B) is the interesterified sample showing that the 18:2 from the soy TAG interesterified with the fCO FA and reducing the dramatically abundance of the native TAG from fCO.

These ESI-MS infusion experiments are intended to demonstrate the chemistry and basis analysis. Each of the labeled TGA peaks in FIGS. 5 & 6 represent isobars of regioisomers as well as chain length isomers as shown. MS/MS methods to resolve these overlaps are readily available and would be facilitated by knowledge of the FA present (see FIG. 4). Once TAG mixtures such as those shown are characterized quantitatively they can serve as standards to provide response factors for calibration of any similar mixture.

One consideration is whether the randomization can be developed using a particular procedure to produce predictable GL profiles. Numerous publications in the lipid chemistry literature investigate and show that random distributions of TAG are obtained with NaOCH₃ (Sreenivasan, 1978) and enzymatic interesterification methods (Gibon et al., 2011). While theory and experiment are in accord, these studies were accomplished at the industrial scale and must be verified to be fit-for-the(-new)-purpose of creating standards because of the rigor with which standards must be characterized. Moreover, any chemical effects, e.g. steric considerations on regiospecificity originating in the glycerol or from FA structure, that lead to non-randomness should be considered. A series of experiments will be conducted with rigorous reference methods that predictions of random GL profiles are obtained for our optimized method. Should any reproducible non-random effects be found specific experiments involving isotopologue sentinels will be used to characterize them, as necessary.

For acyl groups randomly distributed on a GL, the FA profile should be identical at each of the acyl carrying positions, sn-1,2,3 for TAG and sn-1,2 for PL, and of course identical to the FA profile resulting from total hydrolysis of the GL. For any particular FA profile, the ratio of any two FA will be in fixed proportion.

### Example 3—Randomization/Equilibration of TAG and FAME

An experiment was conducted to demonstrate randomization/equilibration of TAG and FAME. Tri18:1 and methyl-17:0 were mixed (-95:5) and treated with a catalytic amount of NaOCH3. In principle, at equilibrium after reaction, the proportions of 18:1 and 17:0 acyl groups should be identical in TAG and in the FAME. TLC separation of product TAG and FAME show an average difference of 0.11%±0.10% (SD, n=4; not significantly different from 0) between 18:1 in TAG and FAME, demonstrating a high degree of equilibration. This also represents proof that FAME fatty acids can be quantitatively randomized into TAG for the purposes of isotopic sentinels, and the ancillary check that FAME fatty acid profile should reflect TAG profile. This may be the first quantitative demonstration of randomization of TAG and FAME.

### Prophetic Example 4—Evaluating Randomization

The rigor with which the present methods will generate reference GL calibration mixture in part depends on the rigor of reference methods for characterizing randomization. Therefore, tried and true, robust, reproducible reference methods in routine use were selected. Two reference methods, AgTLC and GC-FID will be used to evaluate randomization. While AgTLC is a labor-intensive manual method, it often provides the most reliable results and reference data, and will be subject to routine rigorous quality control. AgTLC enables a visual assessment of separation efficiency and as such is robust unexpected variation in reagent purity, concentration, and operator error. A UHPLC system may be employed. GC-FID methods enable baseline FAME separations verifiable by GC-MS. Moreover, the FID is a robust, predictable, and stable detector, producing accuracy and precision for strong peaks routinely 1-3% RSD.

Strategies to verify randomness. The present randomization methods will be refined by starting with commercially available or easily synthesized TAG molecules of the form AAA, that is, of uniform FA composition. The FA will differ in numbers of db which can then be separated by AgTLC. Randomization predicts the profile of each degree of unsaturation. For example, a binary mixture of SSS and OOO where S=stearic acid, 18:0, and O=oleic acid, 18:1, when randomized yields a four AgTLC bands corresponding to 0, 1, 2, and 3 db. 0 db will be S exclusively, while 3 db will be O exclusively and they will be in the same proportion. 1 db will be an equimolar mixture of OSS, SOS, SSO, where the three position are the sn-1,2,3 in order. 2 db will be OOS, OSO, SOO. The 1 and 2 db bands will be of equal abundance and 3-fold greater abundance than the 1 and 4 db bands. Bands will be scraped, FAME prepared and analyzed. SDs for such analyses are <3% RSD with accuracy at least as good. This will provide a strong test of randomization for simple mixtures.

Previous studies have shown excellent agreement with predicted randomness after interesterification in a similar way (Klemann et al., 1994). However, the purpose of previous work is to modify fat properties, and not to verify randomness for analytical purposes. The present methods will be repeatedly refined using this system, considering kinetics to achieve randomness. NaOCH₃ will be employed for initial studies, and enzymes may later be used.

Once the protocol is refined to obtain satisfactory results with a simple TAG binary system, mixtures covering a range of concentrations will be investigated using AAA type TAG, specifically S, O, L=18:2 (linoleic acid), Ln=18:3(linoleic acid), A=arachidonic acid mixed in proportions 100:10:1:0.1. This will be repeated with longer chain FA E=20:5n-3 and D=22:6n-3 at the 0.1 level as these FA tend to be at lower concentration. Again, proportions of all FA in each band are predictable and form a rigorous test of randomness.

Sample-specific verification of randomization. Once optimized for model systems, randomization must by interesterification procedures will be verified for real samples with complex FA profiles to insure with confidence that all GL are randomly distributed from the FA profile. This may be important for experiment-wise protocols that are envisioned to be implemented. Characteristics of ideal procedures for protocols are that they are straightforward, robust, and self-validating.

Two methods will be used to verify randomization is satisfactory.

GL ratios. As discussed nearby Table 1 above, the ratio or any two GL in a rGL is predictable from their abundance in the corresponding FA profile of the nGL. Ratios of specific GL within the various concentration ranges will be chosen, from high to low abundance FA from the FA profile, to characterize putative rGL and optimize parameters. In milkfat (Table 1), palmitic, oleic, and stearic acids are most abundant while trans (“t”) are least abundant. These will validate first the u-rGL and then be applied to experiment-wise rGL protocols.

Isotopologue sentinels. Two or more GL with isotope-labeled (isotopologue) FA will be included at fixed proportion. Successful randomization will be assessed by sentinel GL that must appear in defined proportions in the rGL mixture. For instance, consider triacylglycerol (TAG) as the GL. Consider two different isotopologues of oleic acid: 17,17,18,18,18-d5-oleic acid (O*) and U-13C-oleic acid (O**). Synthesize TAG consisting of two different TG with O* and O** in all positions—tri-O* and tri-O**. Add these to the GL mixture in equal proportion prior to randomization. After randomization, concentration of the regioisomers O**O*O*, O*O**O* and O*O*O** will be equal. The summed concentration of these isobaric regioisomers will also be equal to the sum of isobaric regioisomers O**O**O*+O**O*O**+O*O**O**. A test of randomization would be the ratio of the signals from electrospray mass spectrometry in MS-1 for the appearance of [O**O*O*] and [O**O**O*], where designates the sum of all regioisomers. Any specific GL where O* appears should have an accompanying GL with O** at the same concentration. Isotopologue FA signals in mass spectrometry are generally sufficiently similar to be an insignificant contributor to bias. This procedure may done with unlabeled FA if instrument response is calibrated by a working standard. Coefficients can be derived directly from these measurements if bias away from strictly random was found.

Two protocols will be developed: 1) High volume u-rGL standards, and 2) Protocols for experiment-wise generation of rGL to evaluate deviations of nGL from randomized distribution.

1) High volume u-rGL standards. For generation of universal standards, a combinatorial chemistry approach will be employed to generate multigram quantities.

An initial set of samples to develop rGL listed below will be broadly useful and the rationale for choosing them illustrates our thinking Only publicly available discard samples, or from retail markets, biobanks, or commercial sources will be obtained. In the case of human or animal samples, those that would not trigger human or vertebrate animal ethics approval may be selected. For example, cow's milk is readily available without an ethics approval. Note that GL tend to be robust in storage since once held at −20° C. they degrade only slowly; samplings need not be treated with special care such as when RNA or proteins are targets. u-nGL standards will be distributed on request to qualified laboratories.

### Prophetic Example 5—Randomization Methods

TAG. NaOCH₃ may be used for randomization (FIGS. 4-6). NaOCH₃ randomization for TAG will be refined and evaluated. Once randomization is verified and complete, parameters and protocols will be optimized for robust ease of use.

Chemical acylation methods may be preferable over enzymatic ones to avoid selectivity inherent in enzymes, though may enzymes may be employed for methods that go to completion, such as quantitative acyl hydrolysis prior to a chemical acylation step. One of the most widely used industrial lipases, Novozyme 435 which is an immobilized Candida antarctica lipase B, produces randomization at the sn-1,3 positions and “near-randomization” at the sn-2 position, the latter implying that extended reaction may reach full randomization (Gruczynska et al., 2013). Advantages are that enzymes operate at moderate temperatures and may overcome potential side product formation.

PL and CL. Enzymes are the primary catalysts used in industrial PL transesterifications. As enzymes are inherently selective, chemical synthetic methods will first be investigated to create rPL.

Stereospecific TAG have been synthesized using chemical methods for studies of structure specificity in mass spectrometry development (Xu and Brenna, 2007). rPL will be synthesized as bulk standards to yield a randomized combinatorial chemistry-like series of PL. Pools of relevant PL will be isolated from readily available sources. Milkfat phospholipids will be investigated, of interest in for infant formulas, as milk fat globule membrane (MFGM; Demmelmair et al., 2018). MFGM contains 5-40% of PC, phosphatidylethanolamine (PE), phosphatidylinositol (PI), and phosphatidylserine (PS) and is available at kilogram quantities at low cost. These PL will be isolated and saponified with NaOH_{(aq)} to yield a FA mixture, and then FA isotopologue sentinels quantitatively added. FA in CCl₄ will be treated with dicyclohexylcarbodiimide; the mixed FA anhydride products will be checked as representative of the nPL by AgTLC and GC-FID. Glycerophosphorylcholine and the mixed FA anhydrides are combined in anhydrous ethanol with antioxidant (e.g. TBHQ) and incubated to yield rPC. An rPC aliquot will be converted to FAME to establish that the rPC FA profile corresponds to the nPC FA profile. AgTLC will be performed to isolate PC by unsaturation, and quantitative FA profiles determined and compared to abundances calculated from rPC from the FA profile. Procedures will be repeated for PE, PI, and PS. Once having developed the method with a low cost but highly relevant set of PL, other relevant PL with dramatically different FA profiles will be investigated.

Methods for CL synthesis are established (Krishna et al., 2004; Lin et al., 2004) including those that enable synthesis of CL with four different FA (Abe et al., 2010). CL with random FA will be synthesized and thus take advantage of procedures with few steps. A three-step synthesis will be employed that applies to saturated and unsaturated acyl chains in reasonable yields (Lin et al., 2004). A diacylglycerol (DAG) mixture will be treated with a bifunctional phosphorylating reagent, o-chlorophenyl dichlorophosphate, to link two DAG with subsequent cleavage of aryl chlorides to yield CL. The initial DAG mixture will be derived from whole brain from mice or cows. While commercially available CL derived from corn-fed cow heart has overwhelmingly linoleic acid (18:2n-6), CL FA profiles vary dramatically. For instance, bovine and mouse brain CL has <10% linoleic acid and in mice varies by strain (Ta et al., 2014; Thompson et al., 1976). Bovine brain will be obtained from slaughterhouses and CL purified according to existing methods (Ioannou et al., 1979). FA will be saponified by NaOH to yield a FA profile characteristic of brain (Ioannou et al., 1979). That FA profile will be converted to FA anhydrides as discussed above, and then acylated into glycerol-3-phosphate. The phosphate group will be removed by a PLC and the CL synthesis performed. Other methods may be employed to prepare CL should these methods prove suboptimal.

Workarounds for u-rGL standards. One consideration is how to deal with reactive water, alcohols, side products. Another consideration is the possible excess over which particular nGL may be above the nGL levels, may be characteristic of particular GL. For instance, brain samples may comprise certain PL with 22:6 paired with 16:0 and 18:1 may be at particularly high excess, indicating high net biology-induced acyl specificity. Three approaches will be used to overcome these issues. 1) Spike the FA profile. FA will be added quantitatively, for instance 22:6 and/or 16:0, to the FA mixture used to synthesize the rGL. In this way, it will be possible to raise the specific GL, if necessary at more than one level, to provide a calibration curve with abundances that reach the higher nGL. 2) Spike the GL itself. For instance, spike with PC-22:6-16:0 itself. In specific cases, the specific GL will be added quantitatively to the rGL. The rGL profiles remain in proportion and calculable from the probabilities. 3) Global standard addition. The method of standard addition requires the quantitative addition of a spike of analyte. Signals from spiked and unspiked along with a knowledge of the spike amount enables calculation of the unknown concentration. The same principle holds for a multiple spike. The nGL will be quantitatively spiked by the rGL or a u-rGL and then the spiked and unspiked samples analyzed. The profile of any analyte will then be calculated from corresponding signals. The presence of sentinel isotopologue FA will aid in calibrating spike levels.

Once methods for creation of universal standards are developed with the test materials above standards will be prepared from the following class of samples, chosen based on their FA profiles.

u-rTAG quantitative libraries.

Human plasma. TAG total levels are routinely determined as a clinical test. Plasma/serum borne TAG are a likely target for translational testing.

Ruminant milk. Ruminant milks are similar in composition because of the biochemical buffering of the rumen microbiota. Cow, sheep, and goat are the common food milk. Cow's milk will be used as a standard for all ruminant milks.

Human milk. As noted the composition of human milk TAG is known to be special compared to ruminant milks and has some FA below significant levels in cow's milk.

Human white and brown adipose. Biobank samples from tissue will be obtained. Cell lines for both white and brown fat (Qin et al., 2016) will also be used and cells will be grown up for extraction.

Human vernix caseosa. Human vernix caseosa is rare (Wang et al., 2018) and has an extraordinary abundance of branched chain FA (Ran-Ressler et al., 2008). This substance is presently stored as discard from previous studies and fresh pooled discard will be used if needed.

Lanolin TAG (sheep skin (wool) fat). Lanolin is an inexpensive commodity that is extracted from freshly sheered sheep wool. It is rich in branched chain FA and can be compared to human vernix.

MCF7 human breast cancer cells; Caco2 human epithelial colorectal adenocarcinoma cells. Cultured cells are key to application of this methodology to molecular biology/biochemistry/chemical biology. MCF7 cells (Park et al., 2018; Park et al., 2016; Park et al., 2016; Park et al., 2015; Park et al., 2011; Park et al., 2009; Park et al., 2012) and Caco2 cells (Wijendran et al., 2015; Yan et al., 2017) are available. Both cell types are common, with Caco2 the most commonly used cells in pharmaceutical research, being a model for drug absorption.

u-rPL and u-rCL. Major PL Groups PC, PE, PS, and PI, and CL Quantitative Libraries Will be Prepared.

Cow milk fat globule membrane (MRGM).

Human plasma and red cells.

Fresh primate brain, gray and white matter. The brain is rich in a unique set of PL uniquely high in long chain PUFA and has no TAG. Gray matter and white matter PL have dramatically different FA profiles (Diau et al., 2005). CL from brain has a dramatically different profile from heart.

Human liver and heart. Liver is a main site of FA remodeling from diet into secreted lipoproteins, while heart has a high flux of FA because it relies on FA for energy.

Cow retinas. Bovine retinas contain very long chain polyunsaturated FA of chain length to 36 carbons and 4-6 double bonds, and are available commercially.

Development of universal chemical reference materials is a specialized task, such as isotopic lipid standards that are required for use by the World Antidoping Agency (WADA) Standards for Laboratories in the accredited doping control labs worldwide (Zhang et al., 2009; Zhang et al., 2012; Zhang et al., 2012).

### Prophetic Example 6—Methods for Experiment-Wise Randomization of GL Classes to Enable Measurement of Arbitrary GL in Specific Lipid Pools

Randomization techniques on an experiment-wise basis will be used to create acyl asymmetry ratios and indexes reflective of net acyl specificity maintained in health and disease GL. Two aliquots of a target GL, one the native GL, the other to be analyzed after randomization, possibly with isotopic sentinels added. Analysis of the two samples under identical conditions will permit calculation of the deviation from randomness for any particular GL by constructing ratios of signals and calibration to absolute concentration (e.g. mmol/dl plasma) by reference to the isotopologue species. Chemistry that is simple and robust enough to be made into a kit will be employed. The range of GL profile and FA profiles that support normal function can then be investigated, as can deviations from normal function. With the increasingly well-developed methods for analysis of proteins, post-translational modifications, and proteoforms, this entirety of membrane composition including deviations from a well-defined reference composition can be defined quantitatively for the first time. The present disclosure may be the first mounted to accomplish calibration and compare relative deviations from random/equilibrium distributions in GL.

The approach will be to start with TAG randomization. The NaOCH₃ method or an analogous technique may be adapted for microchemical/analytical scale randomization. It is envisioned that TAG will be purified by TLC or HPLC at the ˜100 nmol level (80 mg) that would be applicable to cell culture. An aliquot will be treated and converted to FAME for analysis of FA profile, a second aliquot treated with NaOCH₃ to yield rTAG and analyzed aside the nTAG plus an appropriate universal standard rTAG for maximal confidence. Some nTAG may reveal large differences in specific TAG abundances necessitating a second treatment that would require spiking (as discussed above) to optimize quantitative analysis.

PL randomization. PL randomization by interesterification procedures have not been developed for any purpose. Without being bound by any theory, a sequential procedure in which, for instance, PC undergoes base catalyzed hydrolysis, then FA are converted to anhydrides and randomly reacylated to the PC may be effective, but multiple step procedure is less desirable. Jensen & Pitas provide a procedure at the multigram level in which the anhydride is generated directly in the flask used to synthesize the PC (Jensen and Pitas, 1976). This approach may be effective, and will lead directly a to simplified system for both PL and CL randomization. These approaches will be explored to produce rapid, simple, robust, inexpensive reproducible production of rPL and rCL, that can be automated robotically. A robot is now operating for high throughput preparations (ASMS 2019 abstract).

These methods will be applied to develop GL acyl asymmetry parameters in specific GL pools and under specific biomedical and experimental conditions. Numerous indexes derived from FA profiles are in use and in continuous development: T/T. The triene-tetraene ratio T/T=[20:4n-6]/[20:3n-9] has been recognized as the definitive index of essential fatty acid deficiency since the 1970s (see Wene et al., 1975), including by the FDA in considerations of medical products (Teitelbaum et al., 2015). Mead acid (20:3n-9) rises when arachidonic acid (20:4:n-6) synthesis from linoleic acid (18:2n-6) via 18:2→18:3⊖20:3→20:4n-6 is low (Fulco et al., 1959). Similarly, [22:5n-6]/[22:6n-3] is an index of omega-3 deficiency in neural tissue (Greiner et al., 2003). The omega-3 index (O3I) came into use as a clinical test in the last 10 years as a marker for cardiovascular disease risk. O3I=([20:5n-3]+[22:5n-3]+[22:6n-3])/(total FA), that is, the percent of omega-3 long chain FA over the total in red blood cells. O3I has been extensively validated and proponents claim it is a better predictor of CVD than any blood cholesterol measure (Harris et al., 2012; Harris et al., 2013).

GL Asymmetry Indexes. Without being bound by any theory, the rGL distribution represents the lowest entropy/equilibrium distribution of fatty acyl groups on a particular GL. Single parameter measures of GL asymmetry will be developed based on a composite of deviations of the native distribution, for instance according to an elementary parameter such as a mean deviation,

$$A = {\sum }_{i = 1}^{n} ( \frac{{G}_{N}^{i} - {G}_{R}^{i}}{n} )$$

where Gⁱ is the relative abundance of the ith glycerolipid, N is the native mixture, R is randomized mixture. For a completely randomized GL, A=0 because G_{N}ⁱ=G_{R}ⁱ for all i, representing the lowest ΔG state driven almost exclusively by ΔS (entropy). Next, empirical investigation of how various parameters are related fundamentally to membrane composition and eventually function will be performed. For instance, an asymmetry factor expressed as a statistical variance,

$$A = {\sum }_{i = 1}^{n} {( \frac{{G}_{N}^{i} - {G}_{R}^{i}}{n} )}^{2} ,$$

has some possibly mathematical benefits compared to the mean deviation. In some cases, unresolvable overlaps may dictate that GL from GL containing specific FA can be calculated, for instance in the case of retinal HUFA (C22-36; 4-6 db). The A factors may be valuable as measures of the degree of specificity required for function of specific series of GL.

As to the form of the equation, it is not possible to know the best parameter until data are in hand. Conversion of this or a similar parameter to AS is also contemplated, as a fundamental component of the free energy required by the organism to maintain membrane asymmetry for proper function. Membrane dysfunction is envisioned as the organism is unable to maintain minimally necessary asymmetry. This parameter will be investigated via applications, two of which will be described below as exemplary of the type of applications immediately amenable to our proposed analyses.

Applications. The present standards and combinatorial strategies are generally applicable to all GL which are ubiquitous in biology. Additional experiments will be conducted with other cell types (e.g. MCF-7, Caco-2). Initially, two primary applications will be addressed that overcome the potential complex overlap issue: completely (yeast) and partially (retina).

Yeast. Common wild type yeast (Saccharomyces cerevisiae) have the molecular apparatus to make only 10 fatty acids de novo (Table 2; Knittelfelder and Kohlwein, 2017; Knittelfelder and Kohlwein, 2017); this list can be readily distinguished by MS/MS, with MS/MS/MS used to distinguish isomeric 18:1s and 16:1s, knowing they these are the only isomers in the mixture, which has been confirmed by GC-CACI-MS/MS. Yeast readily take up exogenous FA from the media and incorporate them into GL. About 10% of yeast FA are found in TAG, 5% in CL, and the balance in PL, and because yeast are easy to grow and manipulate, the GL lipid amounts are unlimited for the present purposes. 1) With methods developed in SA1, randomized yeast specific TAG, PL, and CL will be prepared with isotopic sentinels. Native and randomized yeast TAG, PL, and CL will be run side-by-side under standardized growth conditions and calibrated as a first demonstration of calibration in an organism. 2) Yeast will then be treated with individual exogenous FA bound to albumen in media, and the randomization repeated to determine the GL into which the exogenous FA are incorporated. A series of branched chain fatty acids will be applied one at a time (iso and anteiso 15:0, and 17:0, iso-14:0, 16:0, 18:0), a series of 18:2s (18:2n-6, 9Z,11E-18:2, 10E,12Z-18:2), 18:3n-3, 20:5n-3 and 22:6n-3. Knowing the exogenous FA, its location will be determined by MS/MS methods. The effects of this exogenous perturbation on the distribution of GL will be examined in all lipid classes in yeast, including the distributions of GL not including the exogenous FA. These types of experiments, including transfection and metabolite studies in yeast and human cell lines, are widely accepted additions to the primate/human PUFA biosynthetic pathway. 3) Yeast growth conditions are easily manipulated; acidic conditions are known to induce lipid remodeling (exchange of FA in GL) and lipid metabolism (Guo et al., 2018). Acidic conditions will be altered as these conditions are known to shift yeast to increase TAG concentrations, initially following previous experiments (Guo et al., 2018). As the pH changes, the alterations in GL will be characterized quantitatively.

TABLE 2
Fatty acids of S. cerevisiae.

| FA | MM |
|---|---|
| 14:0 | 227 |
| 14:1 | 225 |
| 15:0 | 241 |
| 15:1 | 239 |
| 16:0 | 255 |
| 16:1 | 253 |
| 16:1 | 253 |
| 18:0 | 283 |
| 18:1 | 281 |
| 18:1 | 281 |

MM is molecular mass.

Retina and VLCPUFA. Retinal lipids accumulate very long chain PUFA (VLCPUFA; even number 24-36 carbons, 4-6 db). Those with 22-36 carbons (all even numbered) and 6 double bonds (db) appear as 1 isomer (n-3), 24-36 carbon FA with 4 and 5 db appear as 2 isomers (n-3 and n-6) which can be distinguished by collisional dissociation (knowing only these isomers are present). PL with VLPUFA typically appear paired with a 22:6n-3. Most VLCPUFA containing GL in retina will be quantitatively characterized.

Interference with accumulation of these FA via interruption of elongation by ELOVL4 results in retinal degeneration known as Stargardt's Syndrome (Agbaga et al., 2008). ESI-MS/MS/MS fragmentation of PC-derived VLCPUFA yield distinct signals that have overlaps, at most of n-3 vs n-6 db configuration, and otherwise are identical based on chain length and number of db. Aging reduces VLCPUFA, the effect of aging on VLCPUFA will be examined in mice (Hopiavuori et al., 2017). ELOVL4 human-mutant-specific mice have greatly decreased VLCPUFA with a neurological phenotype (Hopiavuori et al., 2018) which will be examined.

The protocol development for TAG profiling and u-rTAGs and experiments in yeast and retinal lipids in parallel will be pursued in parallel. GL profiles will be created and verified to be used as calibration mixtures in TAG, PL, and CL profile analysis by ESI-MS/MS. Methods will be developed to reliably randomize GL classes PL, TAG, and CL on lab scale; international standards will be developed for major classes of GL identified on the basis of their known FA profiles. Methods will be also be developed for experiment-wise randomization of GL classes to enable measurement of arbitrary GL in specific lipid pools. Randomization techniques on an experiment-wise basis will be used to create acyl asymmetry ratios and indexes reflective of net acyl specificity maintained in health and disease GL. These methods will be applied to develop GL acyl asymmetry parameters in specific GL pools and under specific biomedical and experimental conditions.

Two systems that represent a widely unused model and a complex mammalian GL, yeast and retinal lipids, will be investigated. The present methods may lead to the discovery of diseases of acyl asymmetry, and advances in membrane biology via computational modeling using accurate, empirically derived quantitative inputs.

### Example 7—Synthesis and Analyzation of TAG's with 16:0 and 18:1

Expecting synthesis of 8 triacylglycerides that have 4 different masses/peaks in a 1:3:3:1 (A:B:C:D) ratio:

Tripalmitoylglycerol (A; 807.3 g/mol);

1-palmitoyl-2-palmitoyl-3-oleiylglycerol (B; 833.4 g/mol);

1,3-palmitoyl-2-oleylglycerol (B; 833.4 g/mol);

1-oleyl-2-palmitoyl-3-oleylglycerol (B; 833.4 g/mol);

1,3-oleyl-2-palmitoylglycerol (C; 859.4 g/mol);

1-oleyl-2-oleyl-3-palmitoylglycerol (C; 859.4 g/mol);

1-palmitoyl-2-oleyl-3-oleylglycerol (C; 859.4 g/mol);

Trioleylglycerol (D; 885.4 g/mol).

Expecting the 8 PC's to be represented by 4 masses in a 1:3:3:1 ratio, respectively:

TAG(16:0/16:0/16:0) @ 807.3;

TAG(16:0/16:0/18:2) @ 833.4;

PC(18:1/18:1/16:0) @ 859.4;

PC(18:1/18:1/18:1) @ 885.4.

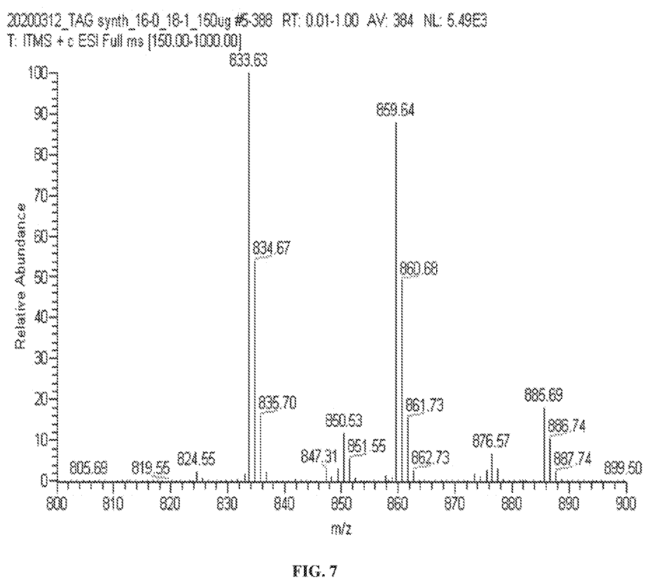

Peak at 807 not observed on spectra (FIG. 7); may be out of range of detection. Interestingly, peaks at 833, 859, and 885 all have two adjacent peaks that each increase by +1. (e.g. 833, 834, 835). These adjacent peaks appear to decrease in intensity by approximately half as the mass increases. Without wishing to be bound by any particular theory, it is hypothesized that this is caused by extra hydrogens. Notably, 8 expected products are not perfectly represented by a 1:3:3:1 ratio. It is hypothesized that increasing the number of moles of glycerol may provide better proportions. Additionally, sodiated TAGS (829, 855, 881, and 907) are not detected.

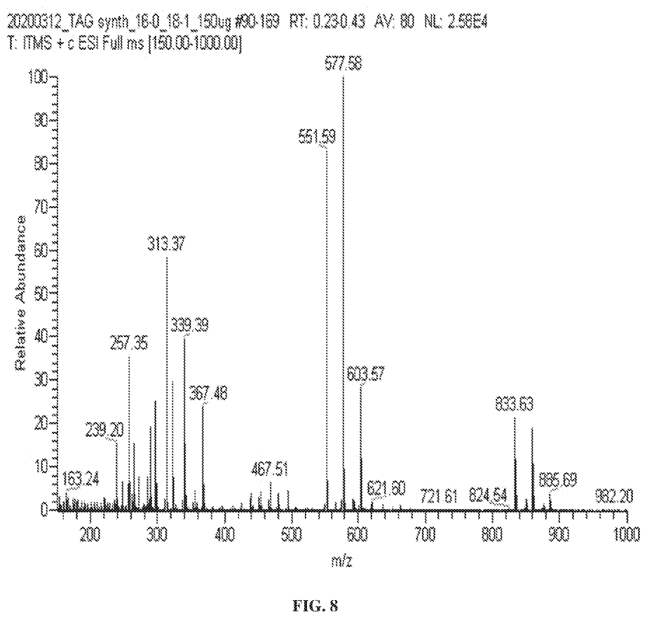

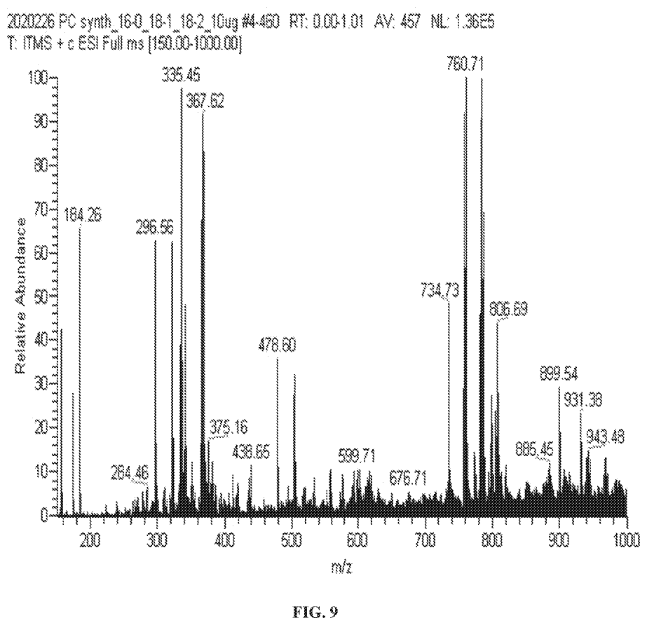

A full spectrum, not zoomed in, is shown in FIG. 8. The relative intensity of the TAG products is small. Mono and diglycerides do not appear to be present in the spectra:

Monopalmitoylglycerol @ 330;

Monooleylglycerol @ 342;

1-oleyl-2-palmitoyl-sn-glycerol @ 594.

In summary, seven out of eight of the expected TAG products appeared in the spectra. Notably, a very small peak at 807 (the missing peak) was observed. It may be that that the relative intensity was too small, and it was out of the range of detection. Products are not demonstrated at a 1:3:3:1 ratio. Increasing the number of moles of glycerol added to reaction may improve the result. No sodiated peaks in spectra despite all previous spectra having these extra peaks. No mono or diglycerides are detected in the spectra.

### Example 8—Synthesis of Randomized Phosphatidyl Cholines (PCs)

A method for synthesis of phosphatidyl cholines (PCs) has been adapted for synthesis of combinatorial PCs (Anankanbil et al., 2018, incorporated herein by reference). Briefly, glycerophosphatidylcholine (GPC) is acylated by any desired mixture of fatty acids.

1-ethyl-3-(3-dimethylaminopropyl)carbodiimide) (EDC), 4-dimethylaminopyridine (DMAP) are used to catalyze the acylation of GPC on Celite® support. Samples of 30 mg PC have been synthesized with mixtures of 2 and 3 fatty acids with excellent results. The method can be easily scaled up to create large amounts of reference standards or down to enable synthesis of randomized PCs from cultures cells. The method also can be used with any phospholipid group, for example phosphatidyl ethanolamine, or phosphatidyl serine or any glycerophospholipid known in biological samples.

### Example 9—Synthesis and Analyzation of PC w/ 16:0, 18:1, and 18:2

Expecting synthesis of 6 phosphatidylcholines in 1:2:1:2:1:2 proportions:

Dipalmitoyl phosphatidylcholine (734 g/mol);

Linoleyl-palmitoyl-sn-glycerol-3-phosphocholine (758 g/mol);

Dilinoleyl phosphatidylcholine (782 g/mol);

Linoleyl-oleic-sn-glycerol-3-phosphocholine (784 g/mol);

Oleoyl-palmitoyl-sn-glycerol-3-phosphocholine (760 g/mol);

Dioleoyl phosphatidylcholine (786 g/mol).

Expecting the 6 PC's to be synthesized in 1:2:2:1:2:1 proportions, respectively

PC(16:0/16:0) @ 734.73;

PC(16:0/18:2) @ 758.74;

PC(16:0/18:1) @ 760.71;

PC(18:2/18:2) @ 782;

PC(18:2/18:1) @ 784;

PC(18:1/18:1) @ 786.

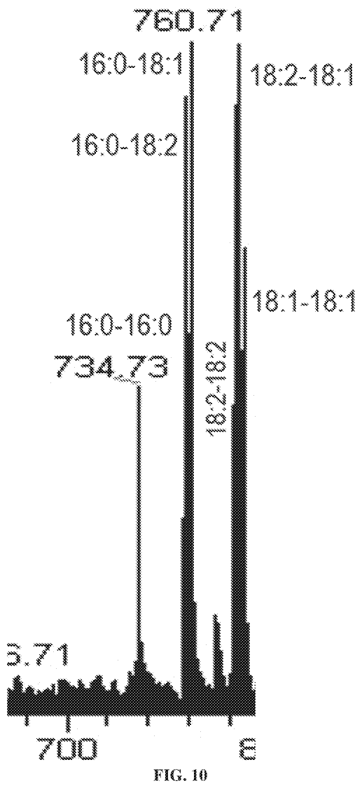

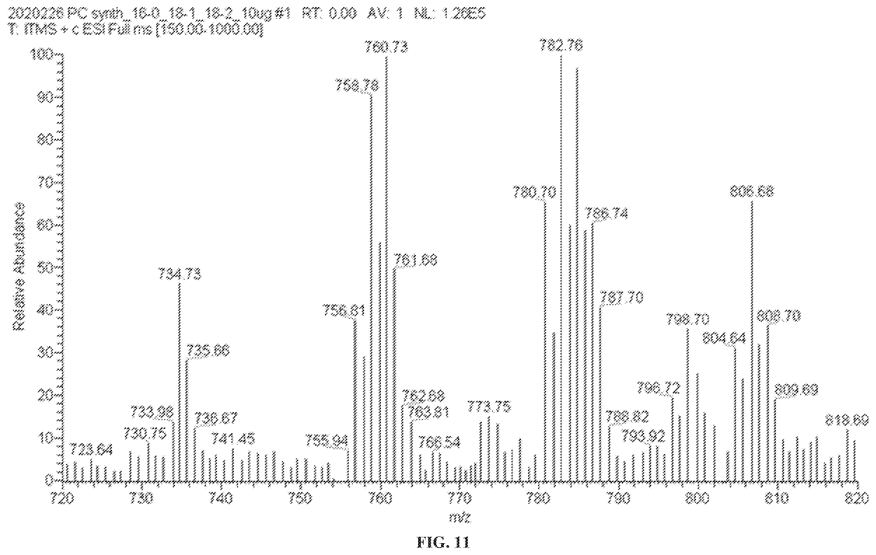

For full spectrum see FIG. 9. For zoom-in of this spectrum at the peaks of interest, see FIG. 10. An expanded view of the zoomed-in spectrum is shown in FIG. 11. It is hypothesized that the extra peaks may be Na-PC, although not every peak is +22amu; some are +21amu (off by one amu). TLC confirmed no TAGs present. In summary, the six expected products do show 1:2:1:2:1:2 proportions. All six anticipated products are found on the spectra, no detectable unreacted GPC backbone at ≈257, and TLC confirmed no TAG's, thus overall the reaction worked well. Six expected products are seen in 1:1:1:2:2:2 proportions, and the proportions are randomized within the experimental error of this screening method.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### IV. References

The following references to the extent that they provide exemplary procedural or other details supplementary to those set forth herein, are specifically incorporated herein by reference.
(ADM), A.D.M. Enzymatically Interesterified Oils and Shortenings. 2018; Available from: https://www.adm.com/products-services/food/oils/interesterified.
Abe, M., S. Kitsuda, S. Ohyama, S. Koubori, M. Murai, and H. Miyoshi, Concise procedure for the synthesis of cardiolipins having different fatty acid combinations. Tetrahedron Letters, 2010. 51(15): p. 2071-2073.
Agbaga, M. P., R. S. Brush, M. N. Mandal, K. Henry, M. H. Elliott, and R. E. Anderson, Role of Stargardt-3 macular dystrophy protein (ELOVL4) in the biosynthesis of very long chain fatty acids. Proc Natl Acad Sci USA, 2008. 105(35): p. 12843-8.
Anankanbil S, Perez B, Banerjee C, Guo Z. New phenophospholipids equipped with multi-functionalities: Regiospecific synthesis and characterization. J Colloid Interface Sci., 523:169-178, 2018.
Brenna et al., Docosahexaenoic and arachidonic acid concentrations in human breast milk worldwide, Am J Clin Nutr, 85(6): p. 1457-64, 2007.
Brenna, J. T., Fatty acid analysis by high resolution gas chromatography and mass spectrometry for clinical and experimental applications, Curr Opin Clin Nutr Metab Care, 16(5): p. 548-54, 2013.
Chojnacka, A., W. Gladkowski, G. Keilbowicz, and W. C., Isolation of egg-yolk phospholipids and enzymatic modification of their acyl chains. Lipid Technology, 2012. 24(2): p. 33-35.
Christensen, E., M. Gronn, T. A. Hagve, B. F. Kase, and B. O. Christophersen, Adrenoleukodystrophy. The chain shortening of erucic acid (22:1(n-9)) and adrenic acid (22:4(n-6)) is deficient in neonatal adrenoleukodystrophy and normal in X-linked adrenoleukodistrophy skin fibroblasts. Biochim Biophys Acta, 1989. 1002(1): p. 79-83.p
Christie, W. W. Preparation of Ester Derivatives of Fatty Acids for Chromatographic Analysis. AOCS Lipid Library 2018 [cited 2018; Available from: http://lipidlibrary.aocs.org/Analysis/content.cfm?ItemNumber=40374 #F.
Cooper, et al., Physiological Consequences of Compartmentalized Acyl-CoA Metabolism. J Biol Chem, 290(33): p. 20023-31, 2015.
De Lue, N., Combinatorial chemistry moves beyond pharmaceuticals. Chemical Innovation, 2001. 31(11): p. 33-39.
Demmelmair, H. and B. Koletzko, Lipids in human milk. Best Pract Res Clin Endocrinol Metab, 2018. 32(1): p. 57-68.
Diau, G. Y., A. T. Hsieh, E. A. Sarkadi-Nagy, V. Wijendran, P. W. Nathanielsz, and J. T. Brenna, The influence of long chain polyunsaturate supplementation on docosahexaenoic acid and arachidonic acid in baboon neonate central nervous system. BMC Med, 2005. 3: p. 11.
Fulco, A. J. and J. F. Mead, Metabolism of essential fatty acids. VIII. Origin of 5,8,11-eicosatrienoic acid in the fat-deficient rat. J Biol Chem, 1959. 234(6): p. 1411-6.
Gibon, V., Enzymatic interestification of oils. Lipid Technology, 2011. 23(12): p. 274-277.
Gomez-Cortes, P., J. T. Brenna, P. Lawrence, and M. A. de la Fuente, Novel characterisation of minor alpha-linolenic acid isomers in linseed oil by gas chromatography and covalent adduct chemical ionisation tandem mass spectrometry. Food Chem, 2016. 200: p. 141-5.
Greiner, R. S., J. N. Catalan, T. Moriguchi, and N. Salem, Jr., Docosapentaenoic acid does not completely replace DHA in n-3 FA-deficient rats during early development. Lipids, 2003. 38(4): p. 431-5.
Gronn, M., E. Christensen, T. A. Hagve, and B. O. Christophersen, The Zellweger syndrome: deficient conversion of docosahexaenoic acid (22:6(n-3)) to eicosapentaenoic acid (20:5(n-3)) and normal delta 4-desaturase activity in cultured skin fibroblasts. Biochim Biophys Acta, 1990. 1044(2): p. 249-54.
Gruczynska, E., D. Kowalska, M. Kozlowska, M. Kowalska, and B. Kowalski, Enzymatic interesterification of a lard and rapeseed oil equal-weight blend. J Oleo Sci, 2013. 62(4): p. 187-93.
Guo, Z. P., S. Khoomrung, J. Nielsen, and L. Olsson, Changes in lipid metabolism convey acid tolerance in Saccharomyces cerevisiae. Biotechnol Biofuels, 2018. 11: p. 297.
Harris, W. S., J. V. Pottala, S. A. Varvel, J. J. Borowski, J. N. Ward, and J. P. McConnell, Erythrocyte omega-3 fatty acids increase and linoleic acid decreases with age: Observations from 160,000 patients. Prostaglandins Leukotrienes and Essential Fatty Acids, 2013.
Harris, W. S., K. F. Kennedy, J. H. O'Keefe, Jr., and J. A. Spertus, Red blood cell fatty acid levels improve GRACE score prediction of 2-yr mortality in patients with myocardial infarction. Int J Cardiol, 2012.
Hites, R., Quantitative Analysis of Triglyceride Mixtures by Mass Spectrometry. Anal Chem, 1970. 14(2): p. 1736-1740.
Hopiavuori, B. R., F. Deak, J. L. Wilkerson, R. S. Brush, N. A. Rocha-Hopiavuori, A. R. Hopiavuori, K. G. Ozan, M. T. Sullivan, J. D. Wren, C. Georgescu, L. Szweda, V. Awasthi, R. Towner, D. M. Sherry, R. E. Anderson, and M. P. Agbaga, Homozygous Expression of Mutant ELOVL4 Leads to Seizures and Death in a Novel Animal Model of Very Long-Chain Fatty Acid Deficiency. Mol Neurobiol, 2018. 55(2): p. 1795-1813.
Hopiavuori, B. R., M. P. Agbaga, R. S. Brush, M. T. Sullivan, W. E. Sonntag, and R. E. Anderson, Regional changes in CNS and retinal glycerophospholipid profiles with age: a molecular blueprint. J Lipid Res, 2017. 58(4): p. 668-680.
Hsieh, J. C., L. Liu, M. Zeilani, S. Ickes, I. Trehan, K. Maleta, C. Craig, C. Thakwalakwa, L. Singh, J. T. Brenna, and M. J. Manary, High-Oleic Ready-to-Use Therapeutic Food Maintains Docosahexaenoic Acid Status in Severe Malnutrition. J Pediatr Gastroenterol Nutr, 2015. 61(1): p. 138-43.
Ioannou, P. V. and B. T. Golding, Cardiolipins: their chemistry and biochemistry. Prog
Lipid Res, 1979. 17(3): p. 279-318.
Jensen, R. G. and C. Lammi-Keefe, The anticarcinogenic conjugated fatty acid c9, t11-c18:2, or rumenic acid, in human milk: amounts and effects. Adv Exp Med Biol, 2001. 501: p. 153-6.
Jensen, R. G. and R. E. Pitas, Synthesis of some acylglycerols and phosphoglycerides. Advances in Lipid Research, 1976. 14: p. 213-1247.
Klemann, L. P., K. A. Aji, M. M. Chrysam, R. P. Amelia, J. M. Henderson, A. S. Huang, M. S. Otterburn, and R. G. Yarger, Random nature of triacylglycerols produced by the catalyzed interesterification of short-and long-chain fatty acid triglycerides. J Agric Food Chem, 1994. 42: p. 442-446.
Knittelfelder, O. L. and S. D. Kohlwein, Derivatization and Gas Chromatography of Fatty Acids from Yeast. Cold Spring Harb Protoc, 2017. 2017(5).
Knittelfelder, O. L. and S. D. Kohlwein, Lipid Extraction from Yeast Cells. Cold Spring Harb Protoc, 2017. 2017(5).
Kramer, J. K., P. W. Parodi, R. G. Jensen, M. M. Mossoba, M. P. Yurawecz, and R. O. Adlof, Rumenic acid: a proposed common name for the major conjugated linoleic acid isomer found in natural products. Lipids, 1998. 33(8): p. 835.
Krishna, U. M., M. U. Ahmad, S. M. Ali, and I. Ahmad, A short, concise route to diphosphatidylglycerol (cardiolipin) and its variants. Lipids, 2004. 39(6): p. 595-600.
Lawrence, P. and J. T. Brenna, Acetonitrile covalent adduct chemical ionization mass spectrometry for double bond localization in non-methylene-interrupted polyene fatty acid methyl esters. Anal Chem, 2006. 78(4): p. 1312-7.
Leng et al., All n-3 PUFA are not the same: MD simulations reveal differences in membrane organization for EPA, DHA and DPA, Biochim Biophys Acta, 1860(5): p. 1125-1134, 2018.
Lin, Z., M. U. Ahmad, S. M. Ali, and I. Ahmad, A new convenient method for the synthesis of cardiolipin. Tetrahedron letters, 2004. 45(37): p. 6923-6925.
Lin, Z., M. U. Ahmad, S. M. Ali, and I. Ahmad, An efficient and novel method for the synthesis of cardiolipin and its analogs. Lipids, 2004. 39(3): p. 285-90.
Liu et al., Higher efficacy of dietary DHA provided as a phospholipid than as a triglyceride for brain DHA accretion in neonatal piglets, J Lipid Res, 55(3): p. 531-9, 2014.
Liu, R., X. Li, and K. S. Lam, Combinatorial chemistry in drug discovery. Curr Opin Chem Biol, 2017. 38: p. 117-126.
Maner, K. M., Characterization of PUFA-containing phospholipids in subcellular fractions: A targeted lipidomics approach by direct infusion ESI-MS/MS/MS, in Nutritional Sciences/Chemistry. 2008, Cornell: Ann Arbor, Mich.
Martinez, M., Abnormal profiles of polyunsaturated fatty acids in the brain, liver, kidney and retina of patients with peroxisomal disorders. Brain Res, 1992. 583(1-2): p. 171-82.
Martinez, M., Severe deficiency of docosahexaenoic acid in peroxisomal disorders: a defect of delta 4 desaturation? Neurology, 1990. 40(8): p. 1292-8.
Mensink et al., The Increasing Use of Interesterified Lipids in the Food Supply and Their Effects on Health Parameters, Adv Nutr, 7(4): p. 719-29, 2016.
Michaud, A. L., G. Y. Diau, R. Abril, and J. T. Brenna, Double bond localization in minor homoallylic fatty acid methyl esters using acetonitrile chemical ionization tandem mass spectrometry. Anal Biochem, 2002. 307(2): p. 348-60.
Murphy, R. C., S. Hammarstrom, and B. Samuelsson, Leukotriene C: a slow-reacting substance from murine mastocytoma cells. Proc Natl Acad Sci USA, 1979. 76(9): p. 4275-9.
Murphy, R. C., T. J. Leiker, and R. M. Barkley, Glycerolipid and cholesterol ester analyses in biological samples by mass spectrometry. Biochim Biophys Acta, 2011. 1811(11): p. 776-83.
O'Donnell-Megaro, A. M., D. M. Barbano, and D. E. Bauman, Survey of the fatty acid composition of retail milk in the United States including regional and seasonal variations. J Dairy Sci, 2011. 94(1): p. 59-65.
Park, H. G., K. S. D. Kothapalli, W. J. Park, C. DeAllie, L. Liu, A. Liang, P. Lawrence, and J. T. Brenna, Palmitic acid (16:0) competes with omega-6 linoleic and omega-3 a-linolenic acids for FADS2 mediated Delta6-desaturation. Biochim Biophys Acta, 2016. 1861(2): p. 91-97.
Park, H. G., M. G. Engel, K. Vogt-Lowell, P. Lawrence, K. S. Kothapalli, and J. T. Brenna, The role of fatty acid desaturase (FADS) genes in oleic acid metabolism: FADS1 Delta? desaturates 11-20:1 to 7,11-20:2. Prostaglandins Leukot Essent Fatty Acids, 2018. 128: p. 21-25.
Park, H. G., P. Lawrence, M. G. Engel, K. Kothapalli, and J. T. Brenna, Metabolic fate of docosahexaenoic acid (DHA; 22:6n-3) in human cells: direct retroconversion of DHA to eicosapentaenoic acid (20:5n-3) dominates over elongation to tetracosahexaenoic acid (24:6n-3). FEBS Lett, 2016. 590(18): p. 3188-94.
Park, H. G., W. J. Park, K. S. Kothapalli, and J. T. Brenna, The fatty acid desaturase 2 (FADS2) gene product catalyzes Delta4 desaturation to yield n-3 docosahexaenoic acid and n-6 docosapentaenoic acid in human cells. FASEB J, 2015. 29(9): p. 3911-9.
Park, W. J., K. S. Kothapalli, H. T. Reardon, P. Lawrence, S. B. Qian, and J. T. Brenna, A novel FADS1 isoform potentiates FADS2-mediated production of eicosanoid precursor fatty acids. J Lipid Res, 2012. 53(8): p. 1502-12.
Park, W. J., K. S. Kothapalli, P. Lawrence, and J. T. Brenna, FADS2 function loss at the cancer hotspot 11q13 locus diverts lipid signaling precursor synthesis to unusual eicosanoid fatty acids. PLoS One, 2011. 6(11): p. e28186.
Park, W. J., K. S. Kothapalli, P. Lawrence, C. Tyburczy, and J. T. Brenna, An alternate pathway to long-chain polyunsaturates: the FADS2 gene product Delta8-desaturates 20:2n-6 and 20:3n-3. J Lipid Res, 2009. 50(6): p. 1195-202.
Qin, X., H. G. Park, J. Y. Zhang, P. Lawrence, G. Liu, N. Subramanian, K. S. Kothapalli, and J. T. Brenna, Brown but not white adipose cells synthesize omega-3 docosahexaenoic acid in culture. Prostaglandins Leukot Essent Fatty Acids, 2016. 104: p. 19-24.
Ran-Ressler, R. R., S. Devapatla, P. Lawrence, and J. T. Brenna, Branched chain fatty acids are constituents of the normal healthy newborn gastrointestinal tract. Pediatr Res, 2008. 64(6): p. 605-9.
Ryan, J. G., No longer “GRAS”: The trans fatty acids debate. Clin Ther, 2014. 36(3): p. 312-4.
Samuelsson, B., P. Borgeat, S. Hammarstrom, and R. C. Murphy, Introduction of a nomenclature: leukotrienes. Prostaglandins, 1979. 17(6): p. 785-7.
Shah, V. P., K. K. Midha, J. W. Findlay, H. M. Hill, J. D. Hulse, I. J. McGilveray, G. McKay, K. J. Miller, R. N. Patnaik, M. L. Powell, A. Tonelli, C. T. Viswanathan, and A. Yacobi, Bioanalytical method validation—a revisit with a decade of progress. Pharm Res, 2000. 17(12): p. 1551-7.
Sreenivasan, Interesterification of Fats., J Am Oil Chem Soc, 55(11): p. 796-805, 1978.
Ta, N. L., X. Jia, M. Kiebish, and T. N. Seyfried, Autosomal dominant inheritance of brain cardiolipin fatty acid abnormality in VM/DK mice: association with hypoxic-induced cognitive insensitivity. Lipids, 2014. 49(1): p. 113-7.
Teitelbaum, D. H., P. Guenter, D. Griebel, S. A. Abrams, S. Bark, M. Baker, K. L. Berry, B. R. Bistrian, J. T. Brenna, D. Bonnot, Y. A. Carpentier, R. J. Deckelbaum, M. Hise, B. Koletzko, J. M. Mirtallo, A. E. Mulberg, R. C. O'Reilly, J. Shaffer, E. von Kleist, G. P. Zaloga, and T. R. Ziegler, Proceedings From FDA/A.S.P.E.N. Public Workshop: Clinical Trial Design for Intravenous Fat Emulsion Products, Oct. 29, 2013. JPEN J Parenter Enteral Nutr, 2015. 39(7): p. 768-86.
Thompson, W. and G. MacDonald, Cytidine diphosphate diglyceride of bovine brain. Positional distribution of fatty acids and analysis of major molecular species. Eur J Biochem, 1976. 65(1): p. 107-11.
Tobias, H. J. and J. T. Brenna, High-volume steroid isotopic standards developed as working standards for gas chromatography-combustion-isotope ratio mass spectrometry. Drug Test Anal, 2018. 10(4): p. 781-785.
Van Pelt, C. K. and J. T. Brenna, Acetonitrile chemical ionization tandem mass spectrometry to locate double bonds in polyunsaturated fatty acid methyl esters. Anal Chem, 1999. 71(10): p. 1981-9.
Van Pelt, C. K., M. C. Huang, C. L. Tschanz, and J. T. Brenna, An octaene fatty acid, 4,7,10,13,16,19,22,25-octacosaoctaenoic acid (28:8n-3), found in marine oils. J Lipid Res, 1999. 40(8): p. 1501-5.
Wang, D. H., J. R. Jackson, C. Twining, L. G. Rudstam, E. Zollweg-Horan, C. Kraft, P. Lawrence, K. Kothapalli, Z. Wang, and J. T. Brenna, Saturated Branched Chain, Normal Odd-Carbon-Numbered, and n-3 (Omega-3) Polyunsaturated Fatty Acids in Freshwater Fish in the Northeastern United States. J Agric Food Chem, 2016.
Wang, D. H., R. Ran-Ressler, J. St Leger, E. Nilson, L. Palmer, R. Collins, and J. T. Brenna, Sea Lions Develop Human-like Vernix Caseosa Delivering Branched Fats and Squalene to the GI Tract. Sci Rep, 2018. 8(1): p. 7478.
Wene, J. D., W. E. Connor, and L. DenBesten, The development of essential fatty acid deficiency in healthy men fed fat-free diets intravenously and orally. J. Clin. Invest., 1975. 56(1): p. 127-134.
Wijendran et al., Efficacy of dietary arachidonic acid provided as triglyceride or phospholipid as substrates for brain arachidonic acid accretion in baboon neonates, Pediatr Res, 51(3): p. 265-72, 2002.
Wijendran, V., J. T. Brenna, D. H. Wang, W. Zhu, D. Meng, K. Ganguli, K. S. Kothapalli, P. Requena, S. Innis, and W. A. Walker, Long-chain polyunsaturated fatty acids attenuate the IL-1 beta-induced proinflammatory response in human fetal intestinal epithelial cells. Pediatr Res, 2015. 78(6): p. 626-33.
Xu, Y. and J. T. Brenna, Atmospheric pressure covalent adduct chemical ionization tandem mass spectrometry for double bond localization in monoene-and diene-containing triacylglycerols. Anal Chem, 2007. 79(6): p. 2525-36.
Yan, Y., Z. Wang, J. Greenwald, K. S. Kothapalli, H. G. Park, R. Liu, E. Mendralla, P. Lawrence, X. Wang, and J. T. Brenna, BCFA suppresses LPS induced IL-8 mRNA expression in human intestinal epithelial cells. Prostaglandins Leukot Essent Fatty Acids, 2017. 116: p. 27-31.
Zhang, J. Y., X. Qin, A. Liang, E. Kim, P. Lawrence, W. J. Park, K. S. D. Kothapalli, and J. T. Brenna, Fads3 modulates docosahexaenoic acid in liver and brain. Prostaglandins Leukot Essent Fatty Acids, 2017. 123: p. 25-32.
Zhang, Q., W. X. Wu, P. W. Nathanielsz, and J. T. Brenna, Distribution of arachidonic, eicosapentaenoic, docosahexaenoic and related fatty acids in ovine endometrial phospholipids in late gestation and labor. Prostaglandins Leukot Essent Fatty Acids, 1995. 53(3): p. 201-9.
Zhang, Y., H. J. Tobias, and J. T. Brenna, Highly sensitive and selective analysis of urinary steroids by comprehensive two-dimensional gas chromatography combined with positive chemical ionization quadrupole mass spectrometry. Analyst, 2012. 137(13): p. 3102-10.
Zhang, Y., H. J. Tobias, and J. T. Brenna, Steroid isotopic standards for gas chromatography-combustion isotope ratio mass spectrometry (GCC-IRMS). Steroids, 2009. 74(3): p. 369-78.
Zhang, Y., H. J. Tobias, G. L. Sacks, and J. T. Brenna, Calibration and data processing in gas chromatography combustion isotope ratio mass spectrometry. Drug Test Anal, 2012. 4(12): p. 912-22.

1. A calibration composition of recombinant glycerolipids (GLs) comprising a mixture of GLs having a plurality of different FAs and/or polar head groups wherein there is a known proportion of each individual GL.

2. The composition of claim 1, wherein the composition is essentially free of free glycerol and/or free fatty acids.

3. The composition of claim 1, wherein the GLs comprise a triacylglycerol TAG mixture.

4. The composition of claim 1, wherein the GLs comprise a phospholipid (PL) mixture.

5. The composition of claim 4, wherein the PL mixture comprises PLs with phosphocholine, phosphoethanolamine and/or phosphoserine head groups.

6. The composition of claim 1, wherein the GLs comprise a cardiolipin (CL) mixture.

7. The composition of claim 1, comprising at least 100 distinct GLs present in a known proportion.

8. The composition of claim 1, comprising at least 500 distinct GLs present in a known proportion.

9. The composition of claim 1, comprising at least 1,000 distinct GLs present in a known proportion.

10. The composition of claim 1, comprising 1,000 to 10,000 GLs present in a known proportion.

11. The composition of claim 1, wherein at least one of the GLs is labeled.

12. The composition of claim 1, wherein at least one of the FAs present in the GLs is labeled.

13. The composition of claim 11 or 12, wherein the label is an isotopic label.

14. The composition of claim 13, wherein the isotopic label is not radioactive.

15. A kit comprising two or more separately packaged compositions according to claim 1, where said separate compositions each comprise different known proportions the individual GLs.

16. A spiked test sample comprising a first portion having an organic sample having an unknown level of GLs and a second portion having a mixture of GLs having a plurality of different FAs and/or polar head groups where there is a known proportion of each individual GL.

17. A method of obtaining a quantitative GL profile of a test sample comprising performing mass spectrometry on a test sample and a calibration composition according to claim 1; and comparing the GL profile of the test sample to the calibration composition thereby obtaining the quantitative GL profile for the test sample.

18. A method of obtaining a quantitative GL profile of a test sample comprising spiking a first portion of a test sample with a calibration composition according to anyone of claim 1; performing mass spectrometry on the first portion of the spiked test sample and a second portion of the test sample; and comparing the GL profile of the first portion of the spiked test sample to the second portion of the test sample thereby obtaining the quantitative GL profile for the test sample.

19. A method for measuring the amounts of a plurality combinatorial analytes in a test sample comprising:
(a) measuring the quantitative profiles (QP) of substituent moieties of the combinatorial analytes;
(b) reacting the substituent moieties to form a mixture of combinatorial analytes in a manner that preserves the QP in a predictable way to generate a calibration standard;
(c) analyzing the calibration standard and a test sample by the same chromatography and/or spectrometry analysis method; and
(d) comparing the analysis of the calibration standard to the analysis of the test sample to determine the amounts of a plurality combinatorial analytes in the test sample.

20. The method of claim 19, wherein the calibration standard and the test sample are analyzed separately.

21. The method of claim 19, wherein the calibration standard and the test sample are mixed prior to analysis.

22. The method of claim 19, further comprising adding an internal standard that is a known quantity of a substituent moiety before the reacting step (b).

23. The method of claim 22, wherein the internal standard is labeled.

24. The method of claim 23, wherein the label is an isotopic label.

25. The method of claim 22, wherein the internal standard was not previously present in the sample of substituent moieties of the combinatorial analytes of (a).

26. A method of producing a calibration standard comprising:
(a′) obtaining a mixture of a plurality of combinatorial analytes and reacting the mixture to produce the substituent moieties of the combinatorial analytes;
(a) measuring the quantitative profiles (QP) of the substituent moieties of the combinatorial analytes; and
(b) reacting the substituent moieties to form a mixture of combinatorial analytes in a manner that preserves the QP in a predictable way to generate a calibration standard.

27. The method of claim 26, further comprising adding an internal standard that is a known quantity of a substituent moiety before the reacting step (b).

28. The method of claim 27, wherein the internal standard is labeled.

29. The method of claim 28, wherein the label is an isotopic label.

30. The method of claim 27, wherein the internal standard was not previously present in the sample of substituent moieties of the combinatorial analytes of (a).

31. The method of claim 26, further comprising analyzing the calibration standard by a chromatography and/or spectrometry analysis method.

32. The method of claim 31, further comprising spiking the calibration standard with a known quantity of at least first combinatorial analyte.

33. The method of claim 26, wherein the plurality of combinatorial analytes comprises at least one phospholipid group.

34. The method of claim 33, wherein the at least one phospholipid group is glycerophosphatidylcholine (GPC).

35. The method of claim 26, wherein the plurality of combinatorial analytes comprises at least one fatty acid.

36. The method of claim 35, wherein the plurality of combinatorial analytes comprises 2 or 3 fatty acids.

37. The method of claim 26, wherein reacting comprises contacting the mixture with at least one reagent suitable to effect acylation.

38. The method of claim 26, wherein reacting comprises contacting the mixture with 1-ethyl-3-(3-dimethylaminopropyl)carbodiimide) (EDC) and 4-dimethylaminopyridine (DMAP).

39. The method of claim 38, wherein reacting further comprises contacting the mixture with a solid support.

40. The method of claim 39, wherein the solid support is diatomaceous earth.

41. A method for measuring the amounts of a plurality combinatorial analytes in a test sample comprising:
(c′) obtaining a calibration standard comprising a plurality of combinatorial analytes, wherein the proportion of each of the plurality of combinatorial analytes in the standard it known;
(c) analyzing the calibration standard and a test sample by the same chromatography and/or spectrometry analysis method; and
(d) comparing the analysis of the calibration standard to the analysis of the test sample to determine the amounts of a plurality combinatorial analytes in the test sample.

42. The method of claim 41, wherein the calibration standard was produced by a method comprising:
(a′) obtaining a mixture of a plurality of combinatorial analytes and reacting the mixture to produce the substituent moieties of the combinatorial analytes;
(a) measuring the quantitative profiles (QP) of the substituent moieties of the combinatorial analytes; and
(b) reacting the substituent moieties to form a mixture of combinatorial analytes in a manner that preserves the QP in a predictable way to generate a calibration standard.

43. The method of claim 19, wherein the analyzing is by chromatography.

44. The method of claim 43, wherein the analyzing is by gas chromatography (GC) or GC-Flame-ionization detection (GC-FID).

45. The method of claim 19, wherein the analyzing is by spectrometry.

46. The method of claim 45, wherein the analyzing is by Electrospray Ionisation Mass Spectrometry (EI-MS).

47. The method of claim 45, wherein the analyzing is by GC/MS or Gas chromatography-electron ionization mass spectrometry (GC-EIMS).

48. The method of claim 19, wherein the plurality of combinatorial analytes comprise a GL mixture.

49. The method of claim 48, wherein the calibration standard comprises at least 100 distinct GLs present in a known proportion.

50. The method of claim 48, wherein the calibration standard comprises at least 500 distinct GLs present in a known proportion.

51. The method of claim 48, wherein the calibration standard comprises at least 1,000 distinct GLs present in a known proportion.

52. The method of claim 48, wherein the calibration standard comprises 1,000 to 10,000 GLs present in a known proportion.

53. The method of claim 19, wherein the plurality of combinatorial analytes comprises a TAG mixture.

54. The method of claim 19, wherein the plurality of combinatorial analytes comprises a CL mixture.

55. The method of claim 19, wherein the substituent moieties of the combinatorial analytes are fatty acids and a non-combinatorial reactant is glycerol.

56. The method of claim 19, wherein the plurality of combinatorial analytes comprises a polypeptide mixture.

57. The method of claim 19, wherein the plurality of combinatorial analytes comprises a carbohydrate mixture.

58. The method of claim 19, wherein the plurality of combinatorial analytes comprises a combination of carbohydrate and lipid mixture.

59. The method of claim 19, wherein the plurality of combinatorial analytes comprises a carbohydrate and protein mixture.

60. The method of claim 19, wherein the plurality of combinatorial analytes comprises a lipid and protein mixture.
